SEQUENCE LISTING (65 sequences)

SEQ ID NO: 1 - protein, 9 aa, synthetic construct
QEVRTISAL

SEQ ID NO: 2 - protein, 9 aa, synthetic construct
QKVRTISAL

SEQ ID NO: 3 - DNA, 390 bp, synthetic construct
  source 1..390: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgaccggggacaggtcgtaggagagacccagtacttcgggccaggcacgcggctcctggtgctc

SEQ ID NO: 4 - DNA, 390 bp, synthetic construct
  source 1..390: mol_type = other DNA
atgctgtctctgctgcttctgcttctcggcctgggctctgtgtttagcgccgtgatcagccagaagcctagccgggatatctgccagagaggcaccagcctgaccatccagtgtcaggtggacagccaagtgaccatgatgttctggtacagacagcagcccggccagtctctgacactgatcgccacagccaatcagggcagcgaggccacatacgagagcggcttcgtgatcgacaagttccccatcagcagacccaacctgaccttcagcaccctgaccgtgtccaacatgagccccgaggacagcagcatctacctgtgctctgatagaggccaggtcgtgggcgagacacagtattttggccctggcaccagactgctggtgctg

SEQ ID NO: 5 - protein, 130 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSDRGQVVGETQYFGPGTRLLVL

SEQ ID NO: 6 - protein, 5 aa, synthetic construct
SQVTM

SEQ ID NO: 7 - protein, 7 aa, synthetic construct
ANQGSEA

SEQ ID NO: 8 - protein, 14 aa, synthetic construct
CSDRGQVVGETQYF

SEQ ID NO: 9 - DNA, 399 bp, synthetic construct
  source 1..399: mol_type = other DNA
atgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtggccgcgaaggatagcagctataaattgatcttcgggagtgggaccagactgctggtcaggcct

SEQ ID NO: 10 - DNA, 399 bp, synthetic construct
  source 1..399: mol_type = other DNA
atgctgctggaactgatccctctgctgggaatccacttcgtgctgagaaccgctagagcccagagcgtgacccagccagatatccacatcaccgtgtctgagggcgccagcctcgagctgagatgcaattacagctacggcgccacaccttacctgttttggtacgtgcagagccccggacagggactgcaactgctgctgaagtactttagcggcgacaccctggtgcagggcatcaagggattcgaggccgagttcaagagaagccagagcagcttcaacctgcggaagccctctgtgcattggagtgatgccgccgagtacttctgtgccgtggccgccaaggatagcagctacaagctgatcttcggcagcggcacaaggctgcttgtcagaccc

SEQ ID NO: 11 - protein, 133 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVAAKDSSYKLIFGSGTRLLVRP

SEQ ID NO: 12 - protein, 6 aa, synthetic construct
YGATPY

SEQ ID NO: 13 - protein, 8 aa, synthetic construct
YFSGDTLV

SEQ ID NO: 14 - protein, 14 aa, synthetic construct
CAVAAKDSSYKLIF

SEQ ID NO: 15 - DNA, 531 bp, synthetic construct
  source 1..531: mol_type = other DNA
gaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 16 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - DNA, 531 bp, synthetic construct
  source 1..531: mol_type = other DNA
gaagatctgaacaaggtgttccctccagaggtggccgtgttcgagccttctgaggccgagatctctcacacccagaaagccacactcgtgtgcctggccaccggcttttttcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgtgtcctatcagcagggcgtgctgagcgccacaatcctgtatgagatcctgctgggcaaagccactctgtacgccgtgctggtgtctgccctggtgctgatggccatggtcaagagaaaggacttt

SEQ ID NO: 18 - protein, 177 aa, synthetic construct
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 19 - DNA, 417 bp, synthetic construct
  source 1..417: mol_type = other DNA
cagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 20 - protein, 139 aa, synthetic construct
QNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - DNA, 417 bp, synthetic construct
  source 1..417: mol_type = other DNA
cagaatcctgatcctgccgtgtaccagctgcgggacagcaagagcagcgataagagcgtgtgtctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccctgaggacacattcttcccaagtcctgagagcagctgcgacgtgaagctggtggaaaagagcttcgagactgacaccaacctcaacttccagaacctgagcgtgatcggctttagaatcctcctgctgaaagtggccggctttaacctgctgatgaccctgagactgtggtcctct

SEQ ID NO: 22 - protein, 139 aa, synthetic construct
QNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - DNA, 1872 bp, synthetic construct
  source 1..1872: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgaccggggacaggtcgtaggagagacccagtacttcgggccaggcacgcggctcctggtgctcgaagatctgaacaaggtgttccctccagaggtggccgtgttcgagccttctgaggccgagatctctcacacccagaaagccacactcgtgtgcctggccaccggcttttttcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgtgtcctatcagcagggcgtgctgagcgccacaatcctgtatgagatcctgctgggcaaagccactctgtacgccgtgctggtgtctgccctggtgctgatggccatggtcaagagaaaggactttggcagcggcgccaccaacttcagcctgcttaaacaggcaggcgacgtggaagagaaccccggacctatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtggccgcgaaggatagcagctataaattgatcttcgggagtgggaccagactgctggtcaggcctgaccagaatcctgatcctgccgtgtaccagctgcgggacagcaagagcagcgataagagcgtgtgtctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccctgaggacacattcttcccaagtcctgagagcagctgcgacgtgaagctggtggaaaagagcttcgagactgacaccaacctcaacttccagaacctgagcgtgatcggctttagaatcctcctgctgaaagtggccggctttaacctgctgatgaccctgagactgtggtcctctggatccggggccacaaatttctcactgctgaagcaggccggggacgtcgaggaaaatcccggaccg

SEQ ID NO: 24 - DNA, 1872 bp, synthetic construct
  source 1..1872: mol_type = other DNA
atgctgtctctgctgcttctgcttctcggcctgggctctgtgtttagcgccgtgatcagccagaagcctagccgggatatctgccagagaggcaccagcctgaccatccagtgtcaggtggacagccaagtgaccatgatgttctggtacagacagcagcccggccagtctctgacactgatcgccacagccaatcagggcagcgaggccacatacgagagcggcttcgtgatcgacaagttccccatcagcagacccaacctgaccttcagcaccctgaccgtgtccaacatgagccccgaggacagcagcatctacctgtgctctgatagaggccaggtcgtgggcgagacacagtattttggccctggcaccagactgctggtgctggaagatctgaacaaggtgttccctccagaggtggccgtgttcgagccttctgaggccgagatctctcacacccagaaagccacactcgtgtgcctggccaccggcttttttcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgtgtcctatcagcagggcgtgctgagcgccacaatcctgtatgagatcctgctgggcaaagccactctgtacgccgtgctggtgtctgccctggtgctgatggccatggtcaagagaaaggactttggcagcggcgccaccaacttcagcctgcttaaacaggcaggcgacgtggaagagaaccccggacctatgctgctggaactgatccctctgctgggaatccacttcgtgctgagaaccgctagagcccagagcgtgacccagccagatatccacatcaccgtgtctgagggcgccagcctcgagctgagatgcaattacagctacggcgccacaccttacctgttttggtacgtgcagagccccggacagggactgcaactgctgctgaagtactttagcggcgacaccctggtgcagggcatcaagggattcgaggccgagttcaagagaagccagagcagcttcaacctgcggaagccctctgtgcattggagtgatgccgccgagtacttctgtgccgtggccgccaaggatagcagctacaagctgatcttcggcagcggcacaaggctgcttgtcagacccgaccagaatcctgatcctgccgtgtaccagctgcgggacagcaagagcagcgataagagcgtgtgtctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccctgaggacacattcttcccaagtcctgagagcagctgcgacgtgaagctggtggaaaagagcttcgagactgacaccaacctcaacttccagaacctgagcgtgatcggctttagaatcctcctgctgaaagtggccggctttaacctgctgatgaccctgagactgtggtcctctggatccggggccacaaatttctcactgctgaagcaggccggggacgtcgaggaaaatcccggaccg

SEQ ID NO: 25 - protein, 624 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSDRGQVVGETQYFGPGTRLLVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVAAKDSSYKLIFGSGTRLLVRPDQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGP

SEQ ID NO: 26 - protein, 21 aa, synthetic construct
  REGION 1..3: note = misc_feature - "GSG" is optional
GSGEGRGSLLTCGDVEENPGP

SEQ ID NO: 27 - protein, 22 aa, synthetic construct
  REGION 1..3: note = misc_feature - "GSG" is optional
GSGATNFSLLKQAGDVEENPGP

SEQ ID NO: 28 - protein, 23 aa, synthetic construct
  REGION 1..3: note = misc_feature - "GSG" is optional
GSGQCTNYALLKLAGDVESNPGP

SEQ ID NO: 29 - protein, 25 aa, synthetic construct
  REGION 1..3: note = misc_feature - "GSG" is optional
GSGVKQTLNFDLLKLAGDVESNPGP

SEQ ID NO: 30 - DNA, 13 bp, synthetic construct
  source 1..13: mol_type = other DNA
  misc_feature 1..3: note = "gcc" is optional
gccgccrccatgg

SEQ ID NO: 31 - protein, 115 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSDRGQVVGETQYFGPGTRLLVL

SEQ ID NO: 32 - protein, 114 aa, synthetic construct
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVAAKDSSYKLIFGSGTRLLVRP

SEQ ID NO: 33 - protein, 9 aa, synthetic construct
RTPDSHHSR

SEQ ID NO: 34 - protein, 9 aa, synthetic construct
RPPDSHHSR

SEQ ID NO: 35 - protein, 9 aa, synthetic construct
RLPDSHHSR

SEQ ID NO: 36 - protein, 9 aa, synthetic construct
GLVDEQQEV

SEQ ID NO: 37 - protein, 9 aa, synthetic construct
GLVDEQQKV

SEQ ID NO: 38 - protein, 9 aa, synthetic construct
FRLHNKPTF

SEQ ID NO: 39 - protein, 9 aa, synthetic construct
SRLHNKPTF

SEQ ID NO: 40 - protein, 10 aa, synthetic construct
RLPDSHHSRR

SEQ ID NO: 41 - protein, 10 aa, synthetic construct
RPPDSHHSRR

SEQ ID NO: 42 - protein, 9 aa, synthetic construct
VQMARYGRL

SEQ ID NO: 43 - protein, 9 aa, synthetic construct
VQMARYGRP

SEQ ID NO: 44 - protein, 10 aa, synthetic construct
QQEVRTISAL

SEQ ID NO: 45 - protein, 10 aa, synthetic construct
QQKVRTISAL

SEQ ID NO: 46 - protein, 9 aa, synthetic construct
DEQQEVRTI

SEQ ID NO: 47 - protein, 9 aa, synthetic construct
DEQQKVRTI

SEQ ID NO: 48 - protein, 9 aa, synthetic construct
FIDSYICQV

SEQ ID NO: 49 - protein, 27 aa, synthetic construct
LRKNHSSAQFIDSYICQVCSRGDEDNK

SEQ ID NO: 50 - protein, 9 aa, synthetic construct
REEMEVHEL

SEQ ID NO: 51 - protein, 25 aa, synthetic construct
EDRDRSHREEMEVHELEKSKRALET

SEQ ID NO: 52 - protein, 9 aa, synthetic construct
RMFPNAPYL

SEQ ID NO: 53 - protein, 28 aa, synthetic construct
PSQASSGQARMFPNAPYLPSCLESQPAI

SEQ ID NO: 54 - protein, 10 aa, synthetic construct
KEFEDDIINW

SEQ ID NO: 55 - protein, 28 aa, synthetic construct
TARTLFNQVMEKEFEDDIINWGRIVTIF

SEQ ID NO: 56 - protein, 9 aa, synthetic construct
DYLQYVLQI

SEQ ID NO: 57 - protein, 31 aa, synthetic construct
MTDCEFGYIYRLAQDYLQYVLQIPQPGSGPS

SEQ ID NO: 58 - protein, 9 aa, synthetic construct
VLHDDLLEA

SEQ ID NO: 59 - protein, 29 aa, synthetic construct
AEGLEKLKECVLHDDLLEARRPRAHECLG

SEQ ID NO: 60 - protein, 9 aa, synthetic construct
ISKERAEAL

SEQ ID NO: 61 - protein, 26 aa, synthetic construct
FLFDTQHFISKERAEALMMPLVDQLE

SEQ ID NO: 62 - protein, 9 aa, synthetic construct
CLAVEEVSL

SEQ ID NO: 63 - protein, 25 aa, synthetic construct
NCFRMTDQEAIQDLCLAVEEVSLRK

SEQ ID NO: 64 - protein, 9 aa, synthetic construct
IEDPPFNSL

SEQ ID NO: 65 - protein, 9 aa, synthetic construct
KECVLHDDL

(19) United States
(12) Patent Application Publication
(10) Pub. No.: US 2025/0025499 A1
(43) Pub. Date: Jan. 23, 2025

(54) T CELL IMMUNOTHERAPY FOR HEMATOLOGIC MALIGNANCIES HAVING AN SF3B1 MUTATION

(71) Applicant: FRED HUTCHINSON CANCER CENTER, Seattle, WA (US)

(72) Inventors: Marie BLEAKLEY, Seattle, WA (US); Melinda BIERNACKI, Seattle, WA (US)

(73) Assignee: FRED HUTCHINSON CANCER CENTER, Seattle, WA (US)

(21) Appl. No.: 18/707,096

(22) PCT Filed: Nov. 1, 2022
(86) PCT No.: PCT/US2022/079078

§ 371 Date: May 2, 2024

Related U.S. Application Data
(60) Provisional application No. 63/274,681, filed on Nov. 2, 2021.

(51) Int. Cl. A61K 35/17; A61K 39/00; A61P 35/00; C07K 14/725; C12N 5/0783; C12N 9/22; C12N 15/86

(52) U.S. Cl. CPC A61K 35/17; A61K 39/4611; A61K 39/4632; A61P 35/00; C07K 14/7051; C12N 5/0636; C12N 9/22; C12N 15/86; C12N 2510/00; C12N 2740/15043

(57) ABSTRACT

Provided are compositions and methods of T cell-based immunotherapies targeting hematologic malignancies that contain a mutation in the SF3BI protein.

### CROSS-REFERENCE TO RELATED APPLICATIONS

This application claims the benefit of U.S. Provisional Patent Application No. 63/274,681, filed on Nov. 2, 2021, the contents of which are incorporated by reference in their entirety.

### SEQUENCE LISTING

This application contains a Sequence Listing, which was submitted in XML format via Patent Center, and is hereby incorporated by reference in its entirety. The XML copy, created on Nov. 1, 2022, is named 0703548001WO00.xml and is 67 KB in size.

### TECHNICAL FIELD

The present technology generally relates to treatment of hematologic malignancies using T cell-based immunotherapies that target a mutation in the SF3B1 protein.

### BACKGROUND

Hematologic malignancies are cancers that affect the blood, bone marrow, and lymph nodes, including various types of leukemia, myeloma, and lymphoma, as well as myelodysplastic syndromes (MDS). MDS includes diverse bone marrow disorders in which the bone marrow does not produce enough healthy blood cells. It arises from somatic mutations acquired in early hematopoietic cells, causing cytopenias and predisposing to transformation into difficult-to-treat secondary acute myeloid leukemia (sAML). There are about 10,000 new diagnoses of MDS per year, and in about 33% of the patients, MDS can progress to sAML. MDS is classified into seven subtypes by the World Health Organization based on blood cell counts, the percentage of blasts in the bone marrow, and risks that the disease will turn into sAML. MDS and sAML are susceptible to T cell-mediated killing, and engineered T cell immunotherapies hold promise for its treatment. Specifically, adoptive T cell therapies employing T cells transduced with antigen-specific T cell receptors (TCRs) (TCR-T) generally have little toxicity and are ideal for patients with resistance to traditional treatment. Careful selection of antigen targets is necessary to preserve residual normal hematopoiesis. Spliceosome gene mutations are prevalent in MDS and sAML and arise early in neoplastic cells, making them attractive therapeutic targets for TCR-T technology.

Splicing factor 3B subunit 1 (SF3B1) is a protein encoded by the SF3B1 gene in humans. A variety of different mutations of SF3B1 have been observed in hematologic malignancies, including MDS, acute myeloid leukemia (AML), and chronic lymphocytic leukemia (CLL). Specifically, SF3B1 mutations are driver mutations in 20-30% of all MDS cases. Of the different possible SF3B1 mutations, the SF3B1^{K700E} mutation is present in 10-15% of all MDS cases. Over 30% of MDS patients with ringed sideroblasts, a subtype of MDS, have the SF3B1^{K700E} mutation. At least 5% of patients with sAML rising from MDS have the SF3B1^{K700E} mutation. SF3B1^{K700E} is also found in about 3% of newly diagnosed CLL cases and about 10% of advanced cases.

Many of the hematologic malignancies are resistant to current treatments and thus remain lethal diseases. They are also more common in older adults, who are more likely to have other comorbidities. While allogeneic hematopoietic cell transplantation (HCT) is the only potentially curative therapy for MDS, AML, and CLL, some patients with comorbidities are ineligible for transplantation. In addition, many patients with these diseases who are eligible for transplantation will require the use of lower intensity transplantation regimens, due to age or comorbidities, which increase the risk of post-transplantation relapse. Post-transplantation relapse is difficult to treat and is associated with poor survival. As a result, a need exists for identification and development of novel molecular targets (e.g., new proteins and/or new antigens of known proteins) and/or therapeutic agents for improved treatments. Immunotherapies that enhance a person's own immune system to fight diseases have become an important component of treatment, and SF3B1^{K700E} presents a potential therapeutic target for the development of novel treatments in patients whose cancers have the SF3B1^{K700E} mutation. Particularly, adoptive immunotherapies such as TCR-T therapy targeting specific cancer mutations would eradicate MDS and sAML cells bearing the mutation while preserving normal hematopoiesis and hold promise for hematologic cancer treatment.

### SUMMARY

The present technology is generally related to compositions and methods of T cell-based immunotherapies that target hematologic malignancies containing spliceosome mutations such as SF3B1 mutations, including, but not limited to, SF3B1^{K700E}.

In some aspects, provided are engineered T cell receptors (TCRs) or fragments thereof that specifically bind SF3B1^{K700E}, an epitope of SF3B1^{K700E}, or an SF3B1^{K700E} epitope/human leukocyte antigen (HLA) complex, comprising: (a) an alpha chain variable region comprising one, two, or three complementarity determining regions (CDRs) having amino acid sequences set forth in SEQ ID NOs: 12-14; and/or (b) a beta chain variable region comprising one, two, or three CDRs having amino acid sequences set forth in SEQ ID NOs: 6-8.

In some embodiments, the epitope of SF3B1^{K700E} has an amino acid sequence set forth in SEQ ID NO: 1. In some embodiments, the HLA is HLA-B*40:01.

In some embodiments, the TCRs or fragments thereof comprise an alpha chain variable region comprising an amino acid sequence that is at least about 80% identical to SEQ ID NO: 32; and/or a beta chain variable region comprising an amino acid sequence that is at least about 80% identical to SEQ ID NO: 31.

In some embodiments, the TCRs or fragments thereof comprise an alpha chain constant region comprising an amino acid sequence that is at least about 80% identical to SEQ ID NO: 20 or SEQ ID NO: 22; and/or a beta chain constant region comprising an amino acid sequence that is at least about 80% identical to SEQ ID NO: 16 or SEQ ID NO: 18.

In some embodiments, the TCRs or fragments thereof comprise an amino acid sequence that is at least about 80% identical to SEQ ID NO: 25.

In some aspects, provided are nucleic acids comprising a nucleotide sequence encoding a TCR or a fragment thereof according to various embodiments of the present technology. In some embodiments, the nucleotide sequence is codon-optimized.

In some aspects, provided are nucleic acids comprising a nucleotide sequence that is at least 80% identical to a nucleotide sequence selected from the group consisting of SEQ ID NOs: 3, 4, 9, 10, 15, 17, 19, 21, 23, and 24.

In some aspects, provided are vectors comprising a nucleic acid according to various embodiments of the present technology. In some embodiments, the vector is a plasmid, an adenoviral vector, an adeno-associated viral vector, a retroviral vector, or a lentiviral vector.

In some aspects, provided are viruses comprising a nucleic acid according to various embodiments of the present technology. In some embodiments, the virus is an adenovirus, an adeno-associated virus, a retrovirus, a lentivirus, or a phage.

In some aspects, provided are compositions comprising a vector or a virus according to various embodiments of the present technology. In some embodiments, the composition further comprises a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a clustered regularly interspaced short palindromic repeat (CRISPR)-associated transposase.

In some aspects, provided are host cells expressing a TCR or a fragment thereof, comprising a nucleic acid, and/or comprising a vector, according to various embodiments of the present technology. In some embodiments, the host cell is a T cell. In some embodiments, the T cell is a CD8+ T cell. In some embodiments, the host cell is an autologous cell. In some embodiments, the host cell is an allogeneic cell. In some embodiments, the host cell is differentiated from an embryonic stem cell (ESC) or an induced pluripotent stem cell (iPSC). In some embodiments, the host cell is a primary cell.

In some embodiments, the host cell is modified to have reduced or eliminated expression of an endogenous TCR. In some embodiments, the host cell has knockout of one or more endogenous TCR genes. In some embodiments, the one or more endogenous TCR genes are selected from the group consisting of TRAC, TRBC1, and TRBC2.

In some embodiments, the host cell is modified to have reduced or eliminated expression of one or more major histocompatibility complex (MHC) class I molecules selected from the group consisting of HLA-A, HLA-B, and HLA-C. In some embodiments, the host cell is modified to express HLA-E and/or HLA-G.

In some aspects, provided are pharmaceutical compositions comprising a host cell according to various embodiments of the present technology.

In some aspects, provided are methods of treating an SF3B1^{K700E}-positive cancer in a subject in need thereof, comprising administering to the subject a host cell or a pharmaceutical composition according to various embodiments of the present technology.

In some aspects, provided are pharmaceutical compositions comprising a host cell according to various embodiments of the present technology for use in a method of treating an SF3B1^{K700E}-positive cancer in a subject in need thereof.

In some aspects, provided are uses of a host cell according to various embodiments of the present technology in the manufacture of a medicament for treating an SF3B1^{K700E}-positive cancer in a subject in need thereof.

In some embodiments, treating an SF3B1^{K700E}-positive cancer comprises inhibiting cancer cell growth, reducing the number of cancer cells, slowing the progression of cancer, decreasing the likelihood of recurrence, or reducing one or more symptoms associated with the cancer.

In some embodiments, the cancer is a hematologic malignancy, for example, myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

### BRIEF DESCRIPTION OF THE DRAWINGS

FIGS. 1A-1B show multiple candidate neoepitopes predicted from recurrent MDS-associated spliceosome mutations. FIG. 1A is a schematic representation of the workflow used for in silico identification of candidate neoantigens. FIG. 1B shows example predictions made using Immune Epitope DataBase (IEDB) consensus algorithms Artificial Neural Network (ANN) and Stabilized Matrix Method (SMM) methods, netMHCpan 4.1, and netCTLpan and summarized graphically. Predicted HLA binders are shown in gray. Predicted cytotoxic T lymphocyte (CTL) binders are shown in black. Epitopes for which the corresponding wild-type (wt) peptide is not predicted to bind HLA are denoted with an asterisk. Candidate epitopes selected for further study (defined as having predicted IC50 of <250 nM or rank <1 by at least two methods, CTL epitope prediction rank <1%, and no predicted HLA binding of the equivalent wild-type peptide) are shown in black with an asterisk.

FIGS. 2A-2G show that the SF3B1^{K700E} neoantigen epitope is immunogenic and primes high-avidity epitope-specific CD8+ T cell clones. FIG. 2A is a schematic of the SF3B1^{K700E} protein and the neoantigen epitope peptide, QEVRTISAL (SEQ ID NO: 1). The corresponding wild-type peptide is QKVRTISAL (SEQ ID NO: 2). FIG. 2B shows that two clones (D1.C4 and D1.C24) were identified after primary in vitro stimulation of CD8+ T cells from an HLA-B*40:01-positive donor with QEVRTISAL (SEQ ID NO: 1) peptide-pulsed antigen-presenting cells (APC) and tested in peptide titration ⁵¹Cr-release cytotoxicity assay (CRA) using autologous lymphoblastoid cell lines (LCL) pulsed with varying peptide concentrations (3 technical replicate experiments). FIG. 2C shows that HLA restriction of D1.C24 (clone 24) was confirmed by testing in CRA against a panel of HLA-typed LCL with single HLA overlap with the original T cell donor. LCL were pulsed with QEVRTISAL (SEQ ID NO: 1) peptide at 1000 ng/ml prior to co-culture (>5 biological replicates per HLA). FIG. 2D shows that high-avidity clone 24 recognizes QEVRTISAL (SEQ ID NO: 1) peptide presented on HLA-B*40:01 but not HLA-B*40:02. Clone 24 was tested in CRA against a panel of HLA-typed LCL that were positive for either HLA-B*40:01 or HLA-B*40:02 and pulsed with QEVRTISAL (SEQ ID NO: 1) peptide (>6 biological replicates per HLA). FIG. 2E shows that clone 24 was tested for recognition of known immunogenic HLA-B*40:01-presented peptides in CRA using autologous LCL pulsed with 1000 ng/mL of each peptide. The tested peptides include REEMEVHEL (SEQ ID NO: 50), IEDPPFNSL (SEQ ID NO: 64), and KECVLHDDL (SEQ ID NO: 65). QEVRTISAL (SEQ ID NO: 1) peptide and the wild-type equivalent QKVRTISAL (SEQ ID NO: 2) were included as controls (3 technical replicate experiments). FIG. 2F shows alanine scanning for clone 24 performed using autologous LCL pulsed with a panel of peptides (1000 ng/ml) with alanine residues substituted at each position, along with two peptides with either a glycine or valine substitution at position 8, a natural alanine residue in the QEVRTISAL (SEQ ID NO: 1) peptide (3 technical replicate experiments). These data were used to identify critical residues for HLA and TCR binding and define the core motif xExRTIxxL. FIG. 2G shows four peptides derived from wild-type human proteins and sharing the xExRTIxxL motif identified using the Scan ProSite tool. To evaluate for cross-recognition of these peptides by clone 24, autologous LCL were pulsed with each peptide (1 ng/ml) and used as targets for clone 24 in CRA (3 technical replicate experiments). For all experiments, mean and standard error of the mean (SEM) are shown.

FIGS. 3A-3G show that the SF3B1^{K700E} neoantigen is naturally processed and presented on neoplastic myeloid cells. FIG. 3A shows percent survival of HNT-34/B*40:01 cells co-cultured with either D1.C24 (clone 24) or an irrelevant neoantigen-specific clone (D2.C32) in flow cytometry-based cytotoxicity assay. Mean and SEM from >3 technical replicates shown. FIG. 3B shows percent survival of SF3B1^{K700E} minigene transduced NB-4 cells co-cultured with either clone D1.C24 (clone 24) or an irrelevant neoantigen-specific clone (D2.C32) in flow cytometry-based cytotoxicity assay. Mean and SEM from >3 technical replicates shown. FIG. 3C shows representative flow plots from CD107a assay demonstrating clone 24 degranulation in response to genotypically SF3B1^{K700E}-positive HLA-B*40:01-positive primary neoplastic myeloid cells (MDS or sAML) but not controls lacking either the mutation or HLA. FIG. 3D shows show that high-avidity clone 24 recognized hematopoietic lines generated from induced pluripotent stem cells (iPSC) derived from a patient with SF3B1^{K700E} MDS in CD107a assay. Controls included an isogenic iPSC-derived line that has wild-type SF3B1 and autologous LCL without or with the SF3B1^{K700E} peptide. Pos, positive; neg, negative; ctrl, control. FIG. 3E shows representative flow plots from CD107a assay demonstrating clone 24 degranulation with genotypically SF3B1^{K700E}-positive HLA-B*40:01-positive MPP-5F line but not SF3B1^{K700E}-negative HLA-B*40:01-positive isogenic control. MPP-5F lines were pre-cultured with interferon-gamma (IFNg) for at least 24 hours. FIG. 3F shows summary data from CD107a assays of clone 24 response to MPP-5F lines under various conditions (>3 biological replicates). Mean and SEM are shown. FIG. 3G shows that MPP-5F lines have low baseline surface HLA class I expression that is reversed by interferon-gamma exposure. Both SF3B1^{K700E}-positive MPP-5F cells and wild-type SF3B1 isogenic controls were evaluated. Class I expression was assessed by flow cytometry after staining with pan-class I antibody after at least 24 hours of pre-culture with IFNg, azacitidine, both, or neither.

FIGS. 4A-4C show that transfer of spliceosome mutation-derived neoantigen-specific TCR confers specificity and function. FIG. 4A shows representative flow plots demonstrating expression of the SF3B1^{K700E}/B*40:01-specific TCR from clone 24 (TCR24) transduced (TD) into primary human CD8+ T cells after CRISPR/Cas9-mediated knock-out of endogenous TCR alpha and beta chains (middle panels) based on staining for the RQR8 transduction marker and with SF3B1^{K700E}/B*40:01-pHLA tetramer. Parental cells and cells not transduced (UTD) were included as control. FIG. 4B shows testing of TCR24 TD T cells for specific lytic activity in peptide titration CRA (technical triplicates). FIG. 4C shows percent survival of HNT-34 cells without and with transduction of HLA-B*40:01 co-cultured with TCR24 TD T cells, parental clone, or controls in flow cytometry-based cytotoxicity assay (>3 technical replicates).

FIG. 5 shows an experimental schematic of testing spliceosome neoantigen-specific TCR-T cells on myeloid neoplasms in vivo. After sublethal irradiation, immunodeficient NOD scid gamma (NSG) variant mice are injected intravenously with neoplastic myeloid cell lines (naturally SF3B1^{K700E}-positive HNT-34 cell line transduced to express HLA-B*40:01, HLA-B*40:01-positive SF3B1^{K700E}-KI NB-4 cells), allowed to engraft for a few weeks, then injected with either spliceosome neoantigen-specific TCR-T cells, irrelevant neoantigen-specific control TCR-T (CBFB-MYH11/B*40:01) cells, or vehicle.

### DETAILED DESCRIPTION

Myelodysplastic syndromes (MDS) are a heterogeneous group of clonal hematopoietic stem cell neoplasms that arise largely from acquisition of somatic mutations in hematopoietic stem/progenitor cells (HSPC) and lead to ineffective hematopoiesis and cytopenias. Furthermore, some MDS will progress into secondary acute myeloid leukemia (sAML), which is often refractory to standard therapies and associated with high morbidity and mortality. Allogeneic hematopoietic cell transplantation (HCT) can be curative for MDS and sAML but is not accessible to all patients due to refractory myeloid disease, comorbidities and/or advanced age. There is an unmet need for safer, more effective therapies for MDS and sAML. The effectiveness of nonmyeloablative HCT and of donor lymphocyte infusion for some MDS and sAML indicates their susceptibility to T cell-mediated killing. T cells can be engineered to express a transgenic receptor, thus redirecting them to recognize a particular target antigen. These receptors can be artificial (e.g., chimeric antigen receptors (CARs)), but natural T cell receptors (TCRs) can also be used. Adoptive T cell therapies employing T cells transduced with antigen-specific TCRs (TCR-T) generally have little toxicity and are suitable even for HCT-ineligible patients with comorbidities.

Mutations in spliceosome genes are prevalent in MDS and sAML. Spliceosome mutations are early events that occur in founding clones and can serve a driver function; this is particularly well established for mutations in SF3B1. Owing to their likely role in transformation, spliceosome mutations are typically clonal and are unlikely to be lost through deletion or transcriptional repression. Neoantigens resulting from such mutations should be optimal for immunotherapeutic targeting. Antigens suitable for targeting in MDS and sAML include neoantigens created from protein-coding mutations found only in malignant cells, such as the spliceosome mutations. Protein products of mutations can be processed into short peptides and presented on the cell surface in the context of human leukocyte antigen (HLA), giving rise to T cell epitopes that are presented on malignant cells but low or absent on normal hematopoietic cells. The clinical importance of T cell responses against neoantigens in antitumor immunity has been demonstrated in multiple settings, including in MDS and AML.

Myeloid malignancies are susceptible to immunologic destruction, but antigen-specific T cell immunotherapies for these diseases are currently limited. In particular, the paucity of neoplastic cells in MDS specimens impedes the identification and validation of targetable neoantigens for the development of immunotherapies for this disorder. Immunotherapies targeting neoantigens created from recurrent protein-coding mutations may selectively eradicate malignant cells and spare their normal counterparts to limit the risk of myeloablation. Accordingly, the present technology provides a T cell immunotherapy-based solution to this problem.

Specifically, spliceosome-derived neoantigens that are present exclusively on malignant cells in MDS and sAML are likely suitable targets for TCR-T, which would eradicate neoplastic cells without impacting normal hematopoiesis. As demonstrated in the working examples, a reverse-immunology strategy using in vitro stimulation of healthy HLA-typed donor CD8+ T cells was employed to identify high-avidity T cells specific for epitopes derived from recurrent spliceosome mutations. Epitopes including SF3B1^{K700E} are naturally processed and presented, including in primary neoplastic myeloid cells. Gene transfer of spliceosome-derived neoantigen-specific TCRs conferred specificity and cytotoxicity to CD8+ T cells which responded to MDS and sAML in vitro and eliminated neoplastic myeloid cells in vivo from cell line-derived xenograft murine models. These data indicate that spliceosome-derived neoantigens are promising targets for TCR-T immunotherapy for individuals with MDS or sAML harboring these mutations.

While the present disclosure is capable of being embodied in various forms, the description below of several embodiments is made with the understanding that the present disclosure is to be considered as an exemplification of the invention and is not intended to limit the invention to the specific embodiments illustrated.

Headings are provided for convenience only and are not to be construed to limit the invention in any manner. Embodiments illustrated under any heading may be combined with embodiments illustrated under any other heading.

### Definitions

Unless otherwise specified, each of the following terms has the meaning set forth in this section.

The indefinite articles “a” and “an” denote at least one of the associated nouns and are used interchangeably with the terms “at least one” and “one or more.” For example, the phrase “a module” means at least one module, or one or more modules.

The conjunctions “or” and “and/or” are used interchangeably.

The term “about,” as used herein when referring to a measurable value, such as an amount or concentration and the like, is meant to encompass variations of 20%, 10%, 5%, 1%, 0.5%, or even 0.1% of the specified amount.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified. Amino acid analogs refer to compounds that have the same basic chemical structure as a naturally occurring amino acid. Such analogs have modified R groups or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

The term “antibody” is used to denote, in addition to natural antibodies, genetically engineered or otherwise modified forms of immunoglobulins or portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)2 fragment, or diabody. An scFv antibody is derived from an antibody by linking the variable regions of the heavy (V_{H}) and light (V_{L}) chains of the immunoglobulin with a short linker peptide. Similarly, a disulfide linked Fv antibody can be generated by linking the V_{H} and V_{L} using an interdomain disulfide bond. On the other hand, sdAbs consist of only the variable region from either the heavy or light chain and usually are the smallest antigen-binding fragments of antibodies. A VHH antibody is the antigen-binding fragment of heavy chain only. A diabody is a dimer of scFv fragment that consists of the V_{H} and V_{L} regions noncovalent connected by a small peptide linker or covalently linked to each other. The antibodies disclosed herein, including those that comprise an immunogenically active portion of an immunoglobulin molecule, retain the ability to bind a specific antigen.

The term “antigen,” as used herein, refers to an immunogenic molecule that provokes an immune response. This immune response may involve antibody production, activation of specific immunologically competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen. Antigens can be presented on the surface of an APC (e.g., macrophage, dendritic cell, or B cell) by MHC molecules for recognition by an immune cell (e.g., T cell). In humans, such MHC molecules include HLA complexes. Specifically, the term “neoantigen,” as used herein, refers to a cancer-specific antigen (i.e., an antigen found on cancer cells but not on non-cancer cells). Neoantigens may arise from one or more cancer-specific alterations in a native protein. Alterations in a native protein that give rise to a neoantigen may be the result of one or more mutations, including for example point mutations, rearrangements, insertions, deletions, or frameshift mutations in the gene encoding the protein or a proximal non-coding region, and/or one or more post-translational modifications such as glycosylation, lipidation, phosphorylation, acetylation, ubiquitination, or SUMOylation.

The term “binding domain” or “binding region” refers to an antibody, a T cell receptor, or portion thereof, that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′) 2 fragments, diabodies, or other synthetic polypeptides selected for their specific ability to bind to a biological molecule, a molecular complex, or other target of interest.

The term “chimeric antigen receptors (CARs),” also known as chimeric T cell receptors or artificial T cell receptors, refers to artificially engineered receptors that combine both antigen-binding and T cell activating functions. CARs may include an extracellular portion comprising a binding domain, such as one obtained or derived from an antibody (e.g., an scFv). The extracellular portion may be linked through a transmembrane domain to one or more intracellular signaling or effector domains. CARs can optionally contain an intracellular costimulatory domain(s). See, e.g., Sadelain et al., Cancer Discov., 3 (4): 388-398 (2013); see also Harris & Kranz, Trends Pharmacol. Sci., 37 (3): 220-230 (2016); Stone et al., Cancer Immunol. Immunother., 63 (11): 1163-1176 (2014). CARs can be introduced to be expressed on the surface of a T cell, so that the T cell can target and kill target cells (e.g., cancer cells) that express the antigen the CAR is designed to bind.

The term “codon-optimized” or “codon optimization,” when referring to a nucleotide sequence, is based on the discovery that the frequency of occurrence of synonymous codons (i.e., codons that code for the same amino acid) in coding nucleotide is biased in different species. Such codon degeneracy allows an identical polypeptide to be encoded by a variety of nucleotide sequences. Codon optimization refers to the process of substituting certain codons in a coding nucleotide sequence with synonymous codons based on the host cell's preference without changing the resulting polypeptide sequence. A variety of codon optimization methods is known in the art, and include, for example, methods disclosed in at least U.S. Pat. Nos. 5,786,464 and 6,114,148.

The terms “complementarity determining region,” and “CDR,” are synonymous with “hypervariable region” or “HVR,” and are known in the art to refer to sequences of amino acids within immunoglobulin (e.g., TCR) variable regions, which, in general, confer antigen specificity and/or binding affinity and are separated from one another in primary amino acid sequence by framework regions. In general, there are three CDRs in each TCR alpha chain variable region and three CDRs in each TCR beta chain variable region. In TCRs, CDR3 is thought to be the main CDR responsible for recognizing processed antigen. In general, CDR1 and CDR2 interact mainly or exclusively with the MHC. CDR1 and CDR2 are encoded within the variable gene segment of a TCR variable region-coding sequence, whereas CDR3 is encoded by the region spanning the variable and joining segments for alpha chain variable region, or the region spanning variable, diversity, and joining segments for beta chain variable region. Thus, if the identity of the variable gene segment is known, the sequences of their corresponding CDR1 and CDR2 can be deduced, e.g., according to a numbering scheme as described herein. Compared with CDR1 and CDR2, CDR3 is typically significantly more diverse due to the addition and loss of nucleotides during the recombination process. TCR variable region sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, a CDR of the present disclosure is identified according to the IMGT numbering scheme (Lefranc et at., Dev. Comp. Immunol. 27:55, 2003; imgt.org/IMGTindex/V-QUEST.php).

The term “conservative substitution,” when referring to amino acid sequences, is recognized in the art as a substitution of one amino acid for another amino acid that has similar properties. A variety of criteria known to persons skilled in the art indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine); and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively.

The term “epitope” includes any molecule, structure, amino acid sequence or protein determinant that is recognized and specifically bound by a cognate binding molecule, such as an antibody or a T cell receptor, or other binding molecule, domain, or protein. Specifically, the term “neoepitope,” as used herein, refers to a cancer-specific epitope (i.e., an epitope found on cancer cells but not on non-cancer cells) that is specifically recognized by a cognate binding molecule as described.

The terms “hematologic malignancy” or “hematologic cancer”, as used herein, refer broadly to any accelerated proliferation of cells, neoplastic or cancerous, diseases, or conditions that affect the blood-forming tissues or cells of the immune system, including, but not limited to, myelodysplastic syndromes, leukemia (e.g., acute lymphocytic leukemia, chronic lymphocytic leukemia, acute myeloid leukemia, chronic myeloid leukemia), myeloma, and lymphoma (e.g., Hodgkin's lymphoma, non-Hodgkin's lymphoma); minimum residual disease following transplantation; multi-drug resistant cancers, primary or secondary malignancies, angiogenesis related to malignancy, or other forms of cancer.

The term “host cell,” as used herein, refers to a cell or microorganism targeted for genetic modification by introduction of a construct or vector carrying a nucleotide sequence for expression of a protein or polypeptide of interest. In certain embodiments, when the protein to be expressed includes an exogenous TCR or fragment thereof, the host cell is usually a T cell.

The term “nucleic acid” or “polynucleotide” refers to a polymeric compound, including covalently linked nucleotides comprising natural subunits (e.g., purine or pyrimidine bases). Purine bases include adenine, and guanine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

The terms “peptide,” “polypeptide,” and “protein” are used interchangeably to refer to a polymer of amino acid residues, and are not limited to a minimum length, though a number of amino acid residues may be specified. Polypeptides may include amino acid residues including natural and/or non-natural amino acid residues. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “subject” refers to a mammalian subject, preferably a human. A “subject in need thereof” refers to a subject who has been diagnosed with cancer, such as hematologic malignancies, or is at an elevated risk of developing cancer. The phrases “subject” and “patient” are used interchangeably herein.

The terms “treat,” “treating,” and “treatment,” as used herein with regard to cancer, refers to alleviating the cancer partially or entirely, inhibiting cancer cell growth, reducing the number of cancer cells, preventing the cancer, decreasing the likelihood of occurrence or recurrence of the cancer, slowing the progression or development of the cancer, or eliminating, reducing, or slowing the development of one or more symptoms associated with the cancer. For example, “treating” may refer to preventing or slowing the existing tumor from growing larger, preventing or slowing the formation or metastasis of cancer, and/or slowing the development of certain symptoms of the cancer. In some embodiments, the term “treat,” “treating,” or “treatment” means that the subject has a reduced number or size of tumor compared to a subject not being administered the treatment. In some embodiments, the term “treat,” “treating,” or “treatment” means that one or more symptoms of the cancer are alleviated in a subject receiving the pharmaceutical compositions as disclosed and described herein, compared to a subject who does not receive such treatment.

A “therapeutically effective amount,” as used herein, is an amount that produces a desired effect in a subject for treating cancer. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the host cell, or the pharmaceutical composition containing the same, and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, NY, 2011, the entire disclosures of which are incorporated by reference herein.

A “vector” refers to a DNA construct containing a nucleic acid molecule that is operably linked to a suitable control sequence capable of effecting the expression of the nucleic acid molecule in a suitable host. Such control sequences may include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself.

### SF3B1^{K700E} Epitopes and TCRs Recognizing the Same

SF3B1 mutations serve as a neoplastic driver for myeloid neoplasms, occurring in 20-30% of all MDS cases and more than 60% of MDS cases with ringed sideroblasts. The SF3B1^{K700E} mutation, in particular, is a commonly seen mutation in hematologic malignancies, affecting 10-15% of MDS patients, 5% of AML patients, and up to 10% of patients with advanced CLL. Due to the high frequencies of the protein-coding SF3B1^{K700E} mutation in hematologic malignancies, it may include epitopes that are useful as antigens for T cell-based immunotherapies for MDS. The terms “SF3B1^{K700E},” “SF3B1 K700E,” “K700E,” and “SF3B1^{mut}” are used interchangeably herein.

T cells are a type of lymphocyte, which develops in the thymus gland and plays a central role in the immune response. There are two major types of T cells: the helper T cell (CD4+) and the cytotoxic T cell (CD8+). T cells express TCRs that can recognize a specific antigen—a molecule (e.g., a peptide or epitope) capable of stimulating an immune response and often produced by cancer cells or viruses. Antigens inside a target cell are bound to class I MHC molecules and brought to the surface of the target cell where they can be recognized by the T cell. When a cytotoxic T cell (i.e., a CD8+ T cell) recognizes an antigen presented at the surface of a target cell through its TCR, the cytotoxic T cell is activated through a cascade of signaling transduction and subsequently initiates killing of the target cell.

In humans, 95% of T cells have a TCR dimer consisting of an alpha chain and a beta chain (encoded by TRA and TRB genes, respectively) linked together by disulfide bonds. In the other 5% of T cells, the TCR consists of delta and gamma chains (encoded by TRD and TRG genes, respectively). Each chain has two extracellular domains: a variable (V) region and a constant (C) region. The constant region is proximal to the cell membrane, followed by a transmembrane region and a short cytoplasmic tail. The variable region recognizes and binds to the antigen/MHC complex. The variable region of both the alpha and beta chains has three CDRs: CDR1, CDR2, and CDR3. When the TCR engages with its specific antigen/MHC complex, the T cell is activated through signal transduction.

Also part of the functional TCR complex is cluster of differentiation 3 (CD3), which is a T cell co-receptor and is involved in activating both the cytotoxic T cell (CD8+ T cells) and T helper cells (CD4+ T cells). In mammals, CD3 may comprise a CD3 gamma chain, a CD3 delta chain, two CD3 epsilon chains, and a homodimer of CD3 zeta chains. As used herein, a “TCR complex” refers to a complex formed by the association of CD3 with TCR. Thus, a TCR complex may be composed of a CD3 gamma chain, a CD3 delta chain, two CD3 epsilon chains, a homodimer of CD3 zeta chains, a TCR alpha chain, and a TCR beta chain. Alternatively, a TCR complex may be composed of a CD3 gamma chain, a CD3 delta chain, two CD3 epsilon chains, a homodimer of CD3 zeta chains, a TCR gamma chain, and a TCR delta chain. When expression of any of the TCR genes is disrupted or eliminated, a functional TCR complex cannot form at the surface of the T cells, which in turn prevents CD3 from locating to the cell surface.

The HLA complex is the human MHC and is specialized to present antigenic peptides to TCRs in humans. The HLA gene complex resides on chromosome 6 and is highly polymorphic, which means that they have many different alleles. In particular, HLA-A, B, and C (corresponding to class I MHC, or MHC I) present peptides from inside the cell as antigens for cytotoxic T cells.

In some aspects, an epitope of SF3B1^{K700E} (also referred to herein as an SF3B1^{K700E} epitope, antigen, or a neoantigen) that is naturally presented by class I MHC (or HLA in humans) molecules at the surface of a cell, such as an SF3B1^{K700E}-positive hematologic cancer cell, is provided in accordance with embodiments of the present technology. The complex formed by the SF3B1^{K700E} epitope and the MHC (or HLA) molecule may be referred to as the SF3B1^{K700E} epitope/MHC complex or the SF3B1^{K700E} epitope/HLA complex. Without intending to be limited by any particular theory, the SF3B1^{K700E} epitope may function as an immunogenic antigen to elicit a cytotoxic T cell response. As shown in further detail in the examples, an SF3B1^{K700E} epitope having the sequence of QEVRTISAL (SEQ ID NO: 1) has strong predicted binding to a specific HLA allele, HLA-B*40:01. The counterpart wild-type sequence, QKVRTISAL (SEQ ID NO: 2), of SF3B1 is not predicted to bind to the HLA-B*40:01 allele, suggesting that the immunogenicity of the SF3B1^{K700E} epitope is specific to cells that contain the SF3B1^{K700E} mutation (i.e., cancer cell-specific). In addition, the identified SF3B1^{K700E} epitope is naturally processed and presented by HLA-B*40:01 on the surface of primary malignant myeloid cells, and SF3B1^{K700E} specific T cells can recognize and kill primary malignant cells bearing the mutant protein. The SF3B1^{K700E} epitope is therefore useful as a cancer-specific target for T cell-mediated recognition and subsequent killing, and as such, may be useful for the development of T cell-based immunotherapies specific to SF3B1^{K700E}-positive cancers.

In some aspects, provided are binding proteins that specifically recognize and/or bind to SF3B1^{K700E}, an epitope of SF3B1^{K700E}, and/or an SF3B1^{K700E} epitope/HLA complex. In some embodiments, the SF3B1^{K700E} epitope is QEVRTISAL (SEQ ID NO: 1). In some embodiments, the HLA is HLA-B*40:01. In some embodiments, the binding protein is an antibody. In some embodiments, the binding protein is a CAR.

In some embodiments, the binding protein is a TCR or a fragment thereof. Exemplary nucleic acid and amino acid sequences of the beta chain and alpha chains of TCRs that can specifically recognize and/or bind an SF3B1^{K700E} epitope or an SF3B1^{K700E} epitope/HLA complex, including the CDRs, are shown in Table 1. The sequence information of the TCRs specific for the SF3B1^{K700E} epitope may be used to develop engineered TCRs, or fragments thereof, for adoptive cell immunotherapies (e.g., TCR-T) against malignancies expressing the mutant SF3B1^{K700E} protein. In some embodiments, the constant region of the TCR may also be engineered for therapy purposes, for example, by introducing paired cysteine residues within the constant region to ensure pairing of the two chains of the TCR, or by grafting constant regions of a different TCR (e.g., from a different species from human).

TABLE 1
Exemplary sequences of SF3B1^{K700E} TCR beta and alpha chains

| SEQ | | |
|---|---|---|
| ID | | |
| NO: | Sequences | Descriptions |
| 3 | atgctgagtcttctgctccttctcctgggactaggctctgtgttcagtgctgtcatctct | Beta chain variable region |
| | caaaagccaagcagggatatctgtcaacgtggaacctccctgacgatccagtgtcaag | nucleic acid sequence |
| | tcgatagccaagtcaccatgatgttctggtaccgtcagcaacctggacagagcctgac | (wild-type) |
| | actgatcgcaactgcaaatcagggctctgaggccacatatgagagtggatttgtcatt | |
| | gacaagtttcccatcagccgcccaaacctaacattctcaactctgactgtgagcaaca | |
| | tgagccctgaagacagcagcatatatctctgcagcgaccggggacaggtcgtaggag | |
| | agacccagtacttcgggccaggcacgcggctcctggtgctc | |
| 4 | atgctgtctctgctgcttctgcttctcggcctgggctctgtgtttagcgccgtgatcagc | Beta chain variable region |
| | cagaagcctagccgggatatctgccagagaggcaccagcctgaccatccagtgtcagg | nucleic acid sequence |
| | tggacagccaagtgaccatgatgttctggtacagacagcagcccggccagtctctgac | (codon-optimized) |
| | actgatcgccacagccaatcagggcagcgaggccacatacgagagcggcttcgtgat | |
| | cgacaagttccccatcagcagacccaacctgaccttcagcaccctgaccgtgtccaac | |
| | atgagccccgaggacagcagcatctacctgtgctctgatagaggccaggtcgtgggc | |
| | gagacacagtattttggccctggcaccagactgctggtgctg | |
| 5 | MLSLLLLLLGLGSVFSAVISQKPSRDICQRGTSLTIQCQVDSQVTMM | Beta chain variable region |
| | FWYRQQPGQSLTLIATANQGSEATYESGFVIDKFPISRPNLTFSTLTV | amino acid sequence (with |
| | SNMSPEDSSIYLCSDRGQVVGETQYFGPGTRLLVL | leader sequence) |
| 31 | SAVISQKPSRDICQRGTSLTIQCQVDSQVTMMFWYRQQPGQSLTLI | Beta chain variable region |
| | ATANQGSEATYESGFVIDKFPISRPNLTFSTLTVSNMSPEDSSIYLCSD | amino acid sequence |
| | RGQVVGETQYFGPGTRLLVL | (without leader sequence) |
| 6 | SQVTM | Beta chain variable region |
| | | CDR1 amino acid sequence |
| 7 | ANQGSEA | Beta chain variable region |
| | | CDR2 amino acid sequence |
| 8 | CSDRGQVVGETQYF | Beta chain variable region |
| | | CDR3 amino acid sequence |
| 9 | atgctcctggagcttatcccactgctggggatacattttgtcctgagaactgccagagc | Alpha chain variable |
| | ccagtcagtgacccagcctgacatccacatcactgtctctgaaggagcctcactggag | region nucleic acid |
| | ttgagatgtaactattcctatggggcaacaccttatctcttctggtatgtccagtccccc | sequence (wild-type) |
| | ggccaaggcctccagctgctcctgaagtacttttcaggagacactctggttcaaggca | |
| | ttaaaggctttgaggctgaatttaagaggagtcaatcttccttcaatctgaggaaaccc | |
| | tctgtgcattggagtgatgctgctgagtacttctgtgctgtggccgcgaaggatagcag | |
| | ctataaattgatcttcgggagtgggaccagactgctggtcaggcct | |
| 10 | atgctgctggaactgatccctctgctgggaatccacttcgtgctgagaaccgctagag | Alpha chain variable region |
| | cccagagcgtgacccagccagatatccacatcaccgtgtctgagggcgccagcctcg | nucleic acid sequence |
| | agctgagatgcaattacagctacggcgccacaccttacctgttttggtacgtgcagag | (codon-optimized) |
| | ccccggacagggactgcaactgctgctgaagtactttagcggcgacaccctggtgca | |
| | gggcatcaagggattcgaggccgagttcaagagaagccagagcagcttcaacctgc | |
| | ggaagccctctgtgcattggagtgatgccgccgagtacttctgtgccgtggccgccaa | |
| | ggatagcagctacaagctgatcttcggcagcggcacaaggctgcttgtcagaccc | |
| 11 | MLLELIPLLGIHFVLRTARAQSVTQPDIHITVSEGASLELRCNYSYGAT | Alpha chain variable region |
| | PYLFWYVQSPGQGLOLLLKYFSGDTLVQGIKGFEAEFKRSQSSFNLR | amino acid sequence (with |
| | KPSVHWSDAAEYFCAVAAKDSSYKLIFGSGTRLLVRP | leader sequence) |
| 32 | AQSVTQPDIHITVSEGASLELRCNYSYGATPYLFWYVQSPGQGLQLL | Alpha chain variable region |
| | LKYFSGDTLVQGIKGFEAEFKRSQSSFNLRKPSVHWSDAAEYFCAVA | amino acid sequence |
| | AKDSSYKLIFGSGTRLLVRP | (without leader sequence) |
| 12 | YGATPY | Alpha chain variable region |
| | | CDR1 amino acid sequence |
| 13 | YFSGDTLV | Alpha chain variable region |
| | | CDR2 amino acid sequence |
| 14 | CAVAAKDSSYKLIF | Alpha chain variable region |
| | | CDR3 amino acid sequence |
| 15 | gaggacctgaacaaggtgttcccacccgaggtcgctgtgtttgagccatcagaagca | Beta chain constant region |
| | gagatctcccacacccaaaaggccacactggtgtgcctggccacaggcttcttccctg | nucleotide sequence (wild- |
| | accacgtggagctgagctggtgggtgaatgggaaggaggtgcacagtggggtcagc | type) |
| | acggacccgcagcccctcaaggagcagcccgccctcaatgactccagatactgcctg | |
| | agcagccgcctgagggtctcggccaccttctggcagaacccccgcaaccacttccgct | |
| | gtcaagtccagttctacgggctctcggagaatgacgagtggacccaggatagggcca | |
| | aacccgtcacccagatcgtcagcgccgaggcctggggtagagcagactgtggcttta | |
| | cctcggtgtcctaccagcaaggggtcctgtctgccaccatcctctatgagatcctgcta | |
| | gggaaggccaccctgtatgctgtgctggtcagcgcccttgtgttgatggccatggtcaa | |
| | gagaaaggatttc | |
| 16 | EDLNKVFPPEVAVFEPSEAEISHTQKATLVCLATGFFPDHVELSWWV | Beta chain constant region |
| | NGKEVHSGVSTDPQPLKEQPALNDSRYCLSSRLRVSATFWQNPRN | amino acid sequence (wild- |
| | HFRCQVQFYGLSENDEWTQDRAKPVTQIVSAEAWGRADCGFTSVS | type) |
| | YQQGVLSATILYEILLGKATLYAVLVSALVLMAMVKRKDF | |
| 17 | gaagatctgaacaaggtgttccctccagaggtggccgtgttcgagccttctgaggccg | Beta chain constant region |
| | agatctctcacacccagaaagccacactcgtgtgcctggccaccggcttttttcccgat | nucleotide sequence |
| | cacgtggaactgtcttggtgggtcaacggcaaagaggtgcacagcggcgtctgtacc | (cysteine modified and |
| | gatcctcagcctctgaaagagcagcccgctctgaacgacagcagatactgcctgagc | codon-optimized) |
| | agcagactgagagtgtccgccaccttctggcagaaccccagaaaccacttcagatgc | |
| | caggtgcagttctacggcctgagcgagaacgatgagtggacccaggatagagccaa | |
| | gcctgtgacacagatcgtgtctgccgaagcctggggcagagccgattgtggctttacc | |
| | agcgtgtcctatcagcagggcgtgctgagcgccacaatcctgtatgagatcctgctgg | |
| | gcaaagccactctgtacgccgtgctggtgtctgccctggtgctgatggccatggtcaag | |
| | agaaaggacttt | |
| 18 | EDLNKVFPPEVAVFEPSEAEISHTQKATLVCLATGFFPDHVELSWWV | Beta chain constant region |
| | NGKEVHSGVCTDPQPLKEQPALNDSRYCLSSRLRVSATFWQNPRN | amino acid sequence |
| | HFRCQVQFYGLSENDEWTQDRAKPVTQIVSAEAWGRADCGFTSVS | (cysteine modified) |
| | YQQGVLSATILYEILLGKATLYAVLVSALVLMAMVKRKDF | |
| 19 | cagaaccctgaccctgccgtgtaccagctgagagactctaaatccagtgacaagtctg | Alpha chain constant region |
| | tctgcctattcaccgattttgattctcaaacaaatgtgtcacaaagtaaggattctgatg | nucleotide sequence (wild- |
| | tgtatatcacagacaaaactgtgctagacatgaggtctatggacttcaagagcaaca | type) |
| | gtgctgtggcctggagcaacaaatctgactttgcatgtgcaaacgccttcaacaacag | |
| | cattattccagaagacaccttcttccccagcccagaaagttcctgtgatgtcaagctgg | |
| | tcgagaaaagctttgaaacagatacgaacctaaactttcaaaacctgtcagtgattgg | |
| | gttccgaatcctcctcctgaaagtggccgggtttaatctgctcatgacgctgcggctgt | |
| | ggtccagc | |
| 20 | QNPDPAVYQLRDSKSSDKSVCLFTDFDSQTNVSQSKDSDVYITDKTV | Alpha chain constant region |
| | LDMRSMDFKSNSAVAWSNKSDFACANAFNNSIIPEDTFFPSPESSC | amino acid sequence (wild- |
| | DVKLVEKSFETDTNLNFQNLSVIGFRILLLKVAGFNLLMTLRLWSS | type) |
| 21 | cagaatcctgatcctgccgtgtaccagctgcgggacagcaagagcagcgataagagc | Alpha chain constant region |
| | gtgtgtctgttcaccgacttcgacagccagaccaacgtgtcccagagcaaggacagc | nucleotide sequence |
| | gacgtgtacatcaccgataagtgcgtgctggacatgcggagcatggacttcaagagc | (cysteine modified and |
| | aacagcgccgtggcctggtccaacaagagcgatttcgcctgcgccaacgccttcaac | codon-optimized) |
| | aacagcattatccctgaggacacattcttcccaagtcctgagagcagctgcgacgtga | |
| | agctggtggaaaagagcttcgagactgacaccaacctcaacttccagaacctgagcg | |
| | tgatcggctttagaatcctcctgctgaaagtggccggctttaacctgctgatgaccctg | |
| | agactgtggtcctct | |
| 22 | QNPDPAVYQLRDSKSSDKSVCLFTDFDSQTNVSQSKDSDVYITDKC | Alpha chain constant region |
| | VLDMRSMDFKSNSAVAWSNKSDFACANAFNNSIIPEDTFFPSPESS | amino acid sequence |
| | CDVKLVEKSFETDTNLNFQNLSVIGFRILLLKVAGFNLLMTLRLWSS | (cysteine modified) |

In some aspects, provided are engineered TCRs or fragments thereof that specifically bind SF3B1^{K700E}, an epitope thereof, and/or an SF3B1^{K700E} epitope/HLA complex. In some embodiments, the SF3B1^{K700E} epitope is QEVRTISAL (SEQ ID NO: 1). In some embodiments, the HLA is HLA-B*40:01. In some embodiments, the TCRs or fragments thereof are human or humanized. In some embodiments, the TCRs or fragments thereof may be a TCR, a single chain TCR (scTCR), or a binding domain of a TCR.

In some embodiments, the TCRs or fragments thereof comprise a variable region comprising or consisting of an amino acid sequence set forth in SEQ ID NO: 5 or SEQ ID NO: 11, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 5 or SEQ ID NO: 11. In some embodiments, the TCRs or fragments thereof comprise a beta chain variable region comprising or consisting of an amino acid sequence set forth in SEQ ID NO: 5, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 5. In some embodiments, the TCRs or fragments thereof comprise an alpha chain variable region comprising or consisting of an amino acid sequence set forth in SEQ ID NO: 11, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 11.

In some embodiments, the beta and/or alpha chain variable regions of the TCRs or fragments thereof may comprise a leader sequence (also referred to as a leader peptide, a signal sequence, or a signal peptide) at the N terminus, which helps direct the expressed beta and/or alpha chains to the cell surface but is cleaved and therefore not part of the TCR at the cell surface. In certain of these embodiments, the TCRs or fragments thereof comprise a variable region comprising or consisting of an amino acid sequence set forth in SEQ ID NO: 31 or SEQ ID NO: 32, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 31 or SEQ ID NO: 32. In some embodiments, the TCRs or fragments thereof comprise a beta chain variable region comprising or consisting of an amino acid sequence set forth in SEQ ID NO: 31, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 31. In some embodiments, the TCRs or fragments thereof comprise an alpha chain variable region comprising or consisting of an amino acid sequence set forth in SEQ ID NO: 32, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 32.

In some embodiments, the variable region of the beta and/or alpha chains of the TCRs or fragments thereof may comprise three CDRs: CDR1, CDR2, and CDR3. CDR3 usually is the main CDR responsible for recognizing antigen processed by MHC molecules. The CDRs of the variable region can be determined using technologies, software, and/or algorithms known to a person skilled in the art, including, for example, the international ImMunoGeneTics information system (IMGT) (https://www.imgt.org/). In certain of these embodiments, the TCRs or fragments thereof comprise one or more (e.g., one, two, three, four, five, or six) CDRs having amino acid sequences set forth in SEQ ID NOs: 6-8 and 12-14, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 6-8 and 12-14.

In some embodiments, the TCRs or fragments thereof comprise a CDR3 comprising or consisting of an amino acid sequence set forth in SEQ ID NO: 8 or SEQ ID NO: 14, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 8 or SEQ ID NO: 14. In some embodiments, the TCRs, or fragments thereof, comprise a beta chain CDR3 comprising or consisting of an amino acid sequence set forth in SEQ ID NO: 8, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 8. In some embodiments, the TCRs, or fragments thereof, comprise an alpha chain CDR3 comprising or consisting of an amino acid sequence set forth in SEQ ID NO: 14, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 14.

In some embodiments, the TCRs or fragments thereof comprise a beta chain, wherein the beta chain comprises one or more (e.g., one, two, or three) CDRs having amino acid sequences set forth in SEQ ID NOs: 6-8, or amino acid sequences that are at about least 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 6-8.

In some embodiments, the TCRs or fragments thereof comprise an alpha chain, wherein the alpha chain comprises one or more (e.g., one, two, or three) CDRs having amino acid sequences set forth in SEQ ID NOs: 12-14, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 12-14.

In some embodiments, the TCRs or fragments thereof comprise a beta chain, wherein the beta chain comprises (1) a CDR1 having an amino acid sequence set forth in SEQ ID NO: 6, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 6; (2) a CDR2 having an amino acid sequence set forth in SEQ ID NO: 7, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 7; and/or (3) a CDR3 having an amino acid sequence set forth in SEQ ID NO: 8, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 8; or any combinations thereof.

In some embodiments, the TCRs or fragments thereof comprise an alpha chain, wherein the alpha chain comprises (1) a CDR1 having an amino acid sequence set forth in SEQ ID NO: 12, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 12; (2) a CDR2 having an amino acid sequence set forth in SEQ ID NO: 13, or an amino acid sequence that is at least about 80% (e.g., at least about 80%, at least about 85%, at least about 90%, at least about 95%, at least about 96%, at least about 97%, at least about 98%, at least about 99%, or about 100% identical) identical to the amino acid sequence set forth in SEQ ID NO: 13; and/or (3) a CDR3 having an amino acid sequence set forth in SEQ ID NO: 14, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 14; or any combinations thereof.

In some embodiments, the TCRs or fragments thereof comprise a constant region comprising or consisting of an amino acid sequence set forth in SEQ ID NO: 16 or SEQ ID NO: 20, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 16 or SEQ ID NO: 20. In some embodiments, the TCRs or fragments thereof comprise a beta chain constant region comprising or consisting of an amino acid sequence set forth in SEQ ID NO: 16, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 16. In some embodiments, the TCRs or fragments thereof comprise an alpha chain constant region comprising or consisting of an amino acid sequence set forth in SEQ ID NO: 20, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 20.

In some embodiments, the beta and/or alpha chain constant regions may be cysteine modified (i.e., modified by introducing cysteine residues at strategic locations within the constant region) to improve pairing of the two chains. In certain of these embodiments, the TCRs or fragments thereof comprise a constant region comprising or consisting of an amino acid sequence set forth in SEQ ID NO: 18 or SEQ ID NO: 22, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 18 or SEQ ID NO: 22. In some embodiments, the TCRs or fragments thereof comprise a beta chain constant region comprising or consisting of an amino acid sequence set forth in SEQ ID NO: 18, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 18. In some embodiments, the TCRs or fragments thereof comprise an alpha chain constant region comprising or consisting of an amino acid sequence set forth in SEQ ID NO: 22, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 22.

In any of those embodiments, the TCRs or fragments thereof may comprise one or more amino acid substitutions, including, for example, conservative substitutions, insertions, and/or deletions from the referenced amino acid sequences.

### Nucleic Acids and Vectors Thereof

In some aspects, provided are nucleic acids comprising a nucleotide sequence encoding a TCR or a fragment thereof according to various embodiments of the present technology. The nucleic acids may be used (for example, in the form of a vector) to transfect or transduce a host cell (e.g., a T cell) so that the host cell would express the encoded exogenous TCR or fragment thereof.

In some embodiments, the nucleic acids comprise a nucleotide sequence encoding a TCR or a fragment thereof, for example, a nucleotide sequence set forth in SEQ ID NO: 3, 9, 15, or 19, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in SEQ ID NO: 3, 9, 15, or 19.

In some embodiments, the nucleic acids comprise a nucleotide sequence that is codon-optimized for a host cell (for example, a human cell) according to techniques known to one of ordinary skill in the art. Codon-optimized sequences include sequences that are partially or fully codon-optimized. In those embodiments, the nucleic acids comprise a nucleotide sequence set forth in SEQ ID NO: 4, 10, 17, or 21, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to a nucleotide sequence set forth in SEQ ID NO: 4, 10, 17, or 21.

In some aspects, provided herein are nucleic acids comprising nucleotide sequences encoding a functional TCR specific to the SF3B1^{K700E} epitope. The nucleic acid may comprise coding sequences for both a beta and an alpha chain (including variable regions and/or constant regions) according to various embodiments of the present technology. The beta chain coding sequence and the alpha chain coding sequence may be connected by a cleavage site (for example, a 2A cleavage site, e.g., a P2A site), so that when expressed inside a host cell (e.g., a T cell), the beta and alpha chains can be expressed as two proteins despite being from one transcript. In any of those embodiments, the nucleotide sequence may be codon-optimized. Exemplary nucleotide and amino acid sequences of the TCRs are shown in Table 2, with annotations of these sequences shown in Table 3.

TABLE 2
Exemplary sequences of TCRs

| SEQ | | |
|---|---|---|
| ID | | |
| NO: | Sequences | Descriptions |
| 23 | atgctgagtcttctgctccttctcctgggactaggctctgtgttcagtgctgtcatctct | TCR nucleic acid sequence |
| | caaaagccaagcagggatatctgtcaacgtggaacctccctgacgatccagtgtcaag | (constant regions are |
| | tcgatagccaagtcaccatgatgttctggtaccgtcagcaacctggacagagcctgac | cysteine modified and |
| | actgatcgcaactgcaaatcagggctctgaggccacatatgagagtggatttgtcatt | codon-optimized; cleave |
| | gacaagtttcccatcagccgcccaaacctaacattctcaactctgactgtgagcaaca | sites are codon-optimized), |
| | tgagccctgaagacagcagcatatatctctgcagcgaccggggacaggtcgtaggag | with a P2A cleavage site |
| | agacccagtacttcgggccaggcacgcggctcctggtgctcgaagatctgaacaagg | connecting the beta and |
| | tgttccctccagaggtggccgtgttcgagccttctgaggccgagatctctcacacccag | alpha chain and a P2A |
| | aaagccacactcgtgtgcctggccaccggcttttttcccgatcacgtggaactgtcttg | cleavage site at the 3′ end |
| | gtgggtcaacggcaaagaggtgcacagcggcgtctgtaccgatcctcagcctctgaa | |
| | agagcagcccgctctgaacgacagcagatactgcctgagcagcagactgagagtgtc | |
| | cgccaccttctggcagaaccccagaaaccacttcagatgccaggtgcagttctacggc | |
| | ctgagcgagaacgatgagtggacccaggatagagccaagcctgtgacacagatcgt | |
| | gtctgccgaagcctggggcagagccgattgtggctttaccagcgtgtcctatcagcag | |
| | ggcgtgctgagcgccacaatcctgtatgagatcctgctgggcaaagccactctgtacg | |
| | ccgtgctggtgtctgccctggtgctgatggccatggtcaagagaaaggactttggcag | |
| | cggcgccaccaacttcagcctgcttaaacaggcaggcgacgtggaagagaaccccg | |
| | gacctatgctcctggagcttatcccactgctggggatacattttgtcctgagaactgcc | |
| | agagcccagtcagtgacccagcctgacatccacatcactgtctctgaaggagcctcac | |
| | tggagttgagatgtaactattcctatggggcaacaccttatctcttctggtatgtccagt | |
| | cccccggccaaggcctccagctgctcctgaagtacttttcaggagacactctggttcaa | |
| | ggcattaaaggctttgaggctgaatttaagaggagtcaatcttccttcaatctgagga | |
| | aaccctctgtgcattggagtgatgctgctgagtacttctgtgctgtggccgcgaaggat | |
| | agcagctataaattgatcttcgggagtgggaccagactgctggtcaggcctgaccag | |
| | aatcctgatcctgccgtgtaccagctgcgggacagcaagagcagcgataagagcgtg | |
| | tgtctgttcaccgacttcgacagccagaccaacgtgtcccagagcaaggacagcgac | |
| | gtgtacatcaccgataagtgcgtgctggacatgcggagcatggacttcaagagcaac | |
| | agcgccgtggcctggtccaacaagagcgatttcgcctgcgccaacgccttcaacaac | |
| | agcattatccctgaggacacattcttcccaagtcctgagagcagctgcgacgtgaagc | |
| | tggtggaaaagagcttcgagactgacaccaacctcaacttccagaacctgagcgtga | |
| | tcggctttagaatcctcctgctgaaagtggccggctttaacctgctgatgaccctgaga | |
| | ctgtggtcctctggatccggggccacaaatttctcactgctgaagcaggccggggacg | |
| | tcgaggaaaatcccggaccg | |
| 24 | atgctgtctctgctgcttctgcttctcggcctgggctctgtgtttagcgccgtgatcagc | TCR nucleic acid sequence |
| | cagaagcctagccgggatatctgccagagaggcaccagcctgaccatccagtgtcagg | (variable regions are |
| | tggacagccaagtgaccatgatgttctggtacagacagcagcccggccagtctctgac | codon-optimized; constant |
| | actgatcgccacagccaatcagggcagcgaggccacatacgagagcggcttcgtgat | regions are cysteine |
| | cgacaagttccccatcagcagacccaacctgaccttcagcaccctgaccgtgtccaac | modified and codon- |
| | atgagccccgaggacagcagcatctacctgtgctctgatagaggccaggtcgtgggc | optimized; cleave |
| | gagacacagtattttggccctggcaccagactgctggtgctggaagatctgaacaag | sites are codon-optimized), |
| | gtgttccctccagaggtggccgtgttcgagccttctgaggccgagatctctcacaccca | with a P2A cleavage site |
| | gaaagccacactcgtgtgcctggccaccggcttttttcccgatcacgtggaactgtctt | connecting the beta and |
| | ggtgggtcaacggcaaagaggtgcacagcggcgtctgtaccgatcctcagcctctga | alpha chain and a P2A |
| | aagagcagcccgctctgaacgacagcagatactgcctgagcagcagactgagagtg | cleavage site at the 3′ end |
| | tccgccaccttctggcagaaccccagaaaccacttcagatgccaggtgcagttctacg | |
| | gcctgagcgagaacgatgagtggacccaggatagagccaagcctgtgacacagatc | |
| | gtgtctgccgaagcctggggcagagccgattgtggctttaccagcgtgtcctatcagc | |
| | agggcgtgctgagcgccacaatcctgtatgagatcctgctgggcaaagccactctgta | |
| | cgccgtgctggtgtctgccctggtgctgatggccatggtcaagagaaaggactttggc | |
| | agcggcgccaccaacttcagcctgcttaaacaggcaggcgacgtggaagagaaccc | |
| | cggacctatgctgctggaactgatccctctgctgggaatccacttcgtgctgagaaccg | |
| | ctagagcccagagcgtgacccagccagatatccacatcaccgtgtctgagggcgcca | |
| | gcctcgagctgagatgcaattacagctacggcgccacaccttacctgttttggtacgtg | |
| | cagagccccggacagggactgcaactgctgctgaagtactttagcggcgacaccctg | |
| | gtgcagggcatcaagggattcgaggccgagttcaagagaagccagagcagcttcaa | |
| | cctgcggaagccctctgtgcattggagtgatgccgccgagtacttctgtgccgtggccg | |
| | ccaaggatagcagctacaagctgatcttcggcagcggcacaaggctgcttgtcagac | |
| | ccgaccagaatcctgatcctgccgtgtaccagctgcgggacagcaagagcagcgata | |
| | agagcgtgtgtctgttcaccgacttcgacagccagaccaacgtgtcccagagcaagg | |
| | acagcgacgtgtacatcaccgataagtgcgtgctggacatgcggagcatggacttca | |
| | agagcaacagcgccgtggcctggtccaacaagagcgatttcgcctgcgccaacgcct | |
| | tcaacaacagcattatccctgaggacacattcttcccaagtcctgagagcagctgcga | |
| | cgtgaagctggtggaaaagagcttcgagactgacaccaacctcaacttccagaacct | |
| | gagcgtgatcggctttagaatcctcctgctgaaagtggccggctttaacctgctgatga | |
| | ccctgagactgtggtcctctggatccggggccacaaatttctcactgctgaagcaggc | |
| | cggggacgtcgaggaaaatcccggaccg | |
| 25 | MLSLLLLLLGLGSVFSAVISQKPSRDICQRGTSLTIQCQVDSQVTMM | TCR amino acid sequence, |
| | FWYRQQPGQSLTLIATANQGSEATYESGFVIDKFPISRPNLTFSTLTV | with P2A cleavage site |
| | SNMSPEDSSIYLCSDRGQVVGETQYFGPGTRLLVLEDLNKVFPPEVA | connecting the beta and |
| | VFEPSEAEISHTQKATLVCLATGFFPDHVELSWWVNGKEVHSGVCT | alpha chain and a P2A |
| | DPQPLKEQPALNDSRYCLSSRLRVSATFWQNPRNHFRCQVQFYGLS | cleavage site at the C- |
| | ENDEWTQDRAKPVTQIVSAEAWGRADCGFTSVSYQQGVLSATILY | terminal |
| | EILLGKATLYAVLVSALVLMAMVKRKDFGSGATNFSLLKQAGDVEE | |
| | NPGPMLLELIPLLGIHFVLRTARAQSVTQPDIHITVSEGASLELRCNYS | |
| | YGATPYLFWYVQSPGQGLOLLLKYFSGDTLVQGIKGFEAEFKRSQSS | |
| | FNLRKPSVHWSDAAEYFCAVAAKDSSYKLIFGSGTRLLVRPDQNPD | |
| | PAVYQLRDSKSSDKSVCLFTDFDSQTNVSQSKDSDVYITDKCVLDM | |
| | RSMDFKSNSAVAWSNKSDFACANAFNNSIIPEDTFFPSPESSCDVKL | |
| | VEKSFETDTNLNFQNLSVIGFRILLLKVAGFNLLMTLRLWSSGSGATN | |
| | FSLLKQAGDVEENPGP | |

TABLE 3
Annotation of TCR sequences of SEQ ID NOs: 23-25

| | Nucleotide | Amino Acid |
|---|---|---|
| | Sequence | Sequence |
| Feature | Position | Position |
| Beta chain variable region | 1-390 | 1-130 |
| Beta chain constant region | 391-921 | 131-307 |
| P2A cleavage site | 922-987 | 308-329 |
| Alpha chain variable region | 988-1386 | 330-462 |
| Alpha chain constant region | 1390-1806 | 464-602 |
| P2A cleavage site | 1807-1872 | 603-624 |

In some embodiments, the nucleic acids comprise a nucleotide sequence encoding a functional TCR, for example, a nucleotide sequence set forth in SEQ ID NO: 23 or SEQ ID NO: 24, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in SEQ ID NO: 23 or SEQ ID NO: 24. In any of these embodiments, the corresponding TCR may comprise an amino acid sequence set forth in SEQ ID NO: 25, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in SEQ ID NO: 25.

In some embodiments, the beta chain coding sequence and the alpha chain coding sequence of the nucleic acid may be connected by a cleavage site, for example, a self-cleaving site. Such a design would allow simultaneous co-expression of two or more separate proteins (e.g., a beta chain and an alpha chain) from one transcript in a host cell. The beta chain coding sequence and the alpha chain coding sequence may be in either 5′ to 3′ order (e.g., 5′-beta chain coding sequence-cleavage site-alpha chain coding sequence-3′, or 5′-alpha chain coding sequence-cleavage site-beta chain coding sequence-3′).

In some embodiments, the self-cleaving site comprises a 2A cleavage site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 4. A glycine-serine-glycine (GSG) linker is optionally added to the N-terminal of a 2A peptide to increase cleavage efficiency. The use of “( )” around a sequence in the present disclosure means that the enclosed sequence is optional.

TABLE 4
Exemplary sequences of 2A peptides

| SEQ | | 2A |
|---|---|---|
| ID NO: | Amino Acid Sequence | Peptide |
| 26 | (GSG)EGRGSLLTCGDVEENPGP | T2A |
| 27 | (GSG)ATNFSLLKQAGDVEENPGP | P2A |
| 28 | (GSG)QCTNYALLKLAGDVESNPGP | E2A |
| 29 | (GSG)VKQTLNFDLLKLAGDVESNPGP | F2A |

In some embodiments, the nucleic acids comprising a nucleotide sequence encoding a TCR or a fragment thereof according to various embodiments of the present technology may be present in the form of a vector (e.g., a plasmid or a viral vector) or packaged into a virus for introduction into a host cell (e.g., a T cell). The vector can be any type of vector suitable for introduction of nucleotide sequences into a host cell, including, for example, plasmids, adenoviral vectors, adeno-associated viral (AAV) vectors, retroviral vectors, lentiviral vectors, phages, and homology-directed repair (HDR)-based donor vectors. The virus can be any type of virus suitable for transducing a host cell and introducing nucleotide sequences into the host cell, including, for example, adenoviruses, adeno-associated viruses, retroviruses, lentiviruses, and phages. In some embodiments, the nucleotide sequence is in a vector (e.g., a viral vector) or a virus which facilitates integration of the nucleotide sequence into a host cell's genome upon introduction into the host cell and thereby replication along with the host genome. In some embodiments, such nucleotide sequence may be present inside a host cell, for example, integrated into the genome of the host cell, for production of a functional TCR or a fragment thereof in the host cell.

In some embodiments, the nucleic acids according to various embodiments of the present technology may be delivered to a host cell via one or more non-viral delivery methods and/or using one or more non-viral vectors, including, but not limited to, physical/mechanical methods, inorganic particles, and synthetic or natural biodegradable particles. Non-limiting examples of physical/mechanical methods include needle injection, ballistic injection, gene gun, electroporation, sonoporation, photoporation, optoporation, magnetofection, and hydroporation. Non-limiting examples of inorganic particles include calcium phosphate, silica, gold, and magnetic particles. Non-limiting examples of synthetic or natural biodegradable particles include polymeric-based non-viral vectors (e.g., poly lactic-co-glycolic acid, poly lactic acid, polyethylene imine, chitosan, dendrimers, polymethacrylates), cationic lipid-based non-viral vectors (e.g., cationic liposomes, cationic emulsions, solid lipid nanoparticles), and peptide-based non-viral vectors (e.g., poly-L-lysine).

In some embodiments, the nucleic acids according to various embodiments of the present technology may be operatively linked to certain regulatory elements of the vector. As known to a skilled artisan, expression vectors are typically engineered to contain polynucleotide sequences that are needed to affect the expression and processing of coding sequences to which they are operatively linked. Expression control sequences may include appropriate transcription initiation, termination, promoter, and enhancer sequences; efficient RNA processing signals, such as splicing and polyadenylation signals; sequences that stabilize cytoplasmic mRNA; sequences that enhance translation efficiency; sequences that enhance protein stability; and possibly sequences that enhance protein secretion. Expression control sequences may be operatively linked if they are contiguous with the gene of interest and expression control sequences that act in trans or at a distance to control the gene of interest.

In some embodiments, the vector may comprise a promoter that drives constitutive gene expression in mammalian cells. Those frequently used promoters include, for example, elongation factor 1 alpha (EF1a) promoter, cytomegalovirus (CMV) immediate-early promoter (Greenaway et al., Gene 18:355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60 (1): 65-74 (1987)), human beta actin promoter, polyubiquitin C gene (UBC) promoter, and CAG promoter (Nitoshi et al., Gene 108:193-199 (1991)).

In some embodiments, the vector may comprise an inducible promoter. Unlike constitutive promoters, inducible promoters can switch between an on and an off state in response to certain stimuli (e.g., chemical agents, temperature, light) and can be regulated in tissue- or cell-specific manners. Non-limiting examples of frequently used inducible promoters include the tetracycline On (Tet-On) system and the tetracycline Off (Tet-Off) system, which utilize tetracycline response elements (TRE) placed upstream of a minimal promoter (e.g., CMV promoter) (Gossen & Bujard, Proc. Natl. Acad. Sci. USA 89 (12): 5547-5551 (1992)). The TRE is made of 7 repeats of a 19-nucleotide tetracycline operator (tetO) sequence and can be recognized by the tetracycline repressor (tetR). In the Tet-Off system, a tetracycline-controlled transactivator (tTA) was developed by fusing the tetR with the activating domain of virion protein 16 of herpes simplex virus. In the absence of tetracycline or its analogs (e.g., doxycycline), the tTA will bind the tetO sequences of the TRE and drives expression; in the presence of tetracycline, the rTA will bind to tetracycline and not to the TRE, resulting in reduced gene expression. Conversely, in the Tet-On system, a reverse transactivator (rtTA) was generated by mutagenesis of amino acid residues important for tetracycline-dependent repression, and the rtTA binds at the TRE and drives gene expression in the presence of tetracycline or doxycycline (Gossen et al., Science 268 (5218): 1766-1769 (1995)). Other examples of inducible promoters include, for example, AlcA, LexA, and Cre.

In some embodiments, the vector may comprise a Kozak consensus sequence, usually upstream of the coding sequence. A Kozak consensus sequence is a nucleic acid motif that functions as the protein translation initiation site in most eukaryotic mRNA transcripts and mediates ribosome assembly and translation initiation. In some embodiments, the Kozak consensus sequence comprises or consists of the sequence of (gcc) gccrccatgg (SEQ ID NO: 30), wherein r is a purine (i.e., a or g).

In some embodiments, the vector may comprise a selection marker that allows identification, detection, selection, enrichment, and/or sorting of the transduced cells. In some embodiments, the selection marker comprises an RQR polypeptide, a truncated low-affinity nerve growth factor (tNGFR), a truncated CD19 (tCD19), a truncated CD34 (tCD34), or any combination thereof.

In some embodiments, the selection marker is an RQR polypeptide. RQR comprises a major extracellular loop of CD20 and two minimal CD34 minimal epitope. In some embodiments, the CD34 minimal epitope is incorporated at the amino terminal position of a CD8 co-receptor stalk domain (Q8). In further embodiments, the CD34 minimal binding site sequence can be combined with a target epitope for CD20 to form a compact marker/suicide gene for T cells (RQR8). This construct allows for the selection of immune cells expressing the construct, with for example, CD34 specific antibody bound to magnetic beads and that utilizes clinically accepted pharmaceutical antibody, rituximab, that allows for the selective deletion of a transduced cell.

In some embodiments, the vector or virus comprising a nucleotide sequence encoding a TCR or a fragment thereof according to various embodiments of the present technology may be present in a composition.

In some embodiments, the composition may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising host cells as disclosed herein further comprise a suitable infusion media.

In some embodiments, the composition containing the vector or virus comprising a nucleotide sequence encoding a TCR or a fragment thereof according to various embodiments of the present technology may further comprises a gene editing system, or a site-directed nuclease, as described for integrating the nucleotide sequence into the genome of the host cell. In some embodiments, the composition containing the vector or virus may be administered in combination with a gene editing system, or a site-directed nuclease, as described for host cell integration.

Introduction of Nucleic Acids and/or Vectors Thereof into Host Cells

In some aspects, the nucleic acids encoding a TCR or a fragment thereof according to various embodiments of the present technology may be introduced into a host cell, so that the host cell would express the encoded exogenous TCR or fragment thereof, for use in adoptive cell therapy.

### Transfection or Transduction of Host Cells

Host cells may be transformed to incorporate the nucleic acids (e.g., in the form of a vector) by any known method in the field, including, for example, viral transduction, calcium phosphate transfection, lipid-mediated transfection, DEAE-dextran, electroporation, microinjection, nucleoporation, liposomes, nanoparticles, or other methods. In some embodiments, the nucleic acids comprising a nucleotide sequence encoding a TCR or a fragment thereof according to various embodiments of the present technology may be in the form of a viral vector or packaged into a virus for introduction into a population of host cells. The virus can be any type of virus suitable for transducing a host cell and introducing nucleotide sequences into the host cell, including, for example, adenoviruses, adeno-associated viruses, retroviruses, lentiviruses, and phages. The transformed host cells can be collected and/or screened using known techniques, and the various subpopulations or combinations thereof can be enriched or depleted by known techniques, such as by affinity binding to antibodies, flow cytometry, fluorescence activated cell sorting (FACS), or immunomagnetic selection. After introduction into the host cell, the nucleic acids can be integrated into the genome of the host cell either through random insertion or through site-directed insertion (knock-in) as described.

In some embodiments, after being introduced into a host cell, the nucleic acids comprising a nucleotide sequence encoding a TCR or a fragment thereof according to various embodiments of the present technology may be integrated in the genome of the host cell.

### Random Insertion

In some embodiments, the nucleic acids encoding a TCR or a fragment thereof according to various embodiments of the present technology are inserted into a random genomic locus of a host cell. As known to a person skilled in the art, viral vectors, including, for example, retroviral vectors, lentiviral vectors, adenoviral vectors, and adeno-associated viral vectors, are commonly used to deliver genetic material into host cells and randomly insert the foreign or exogenous gene into the host cell genome to facilitate stable expression and replication of the gene.

### Site-Directed Insertion (Knock-In)

In some embodiments, the nucleic acids encoding a TCR or a fragment thereof according to various embodiments of the present technology are inserted into a specific genomic locus of the host cell. A number of gene editing methods can be used for inserting a transgene into a specific genomic locus of choice. Gene editing is a type of genetic engineering in which a nucleotide sequence may be inserted, deleted, modified, or replaced in the genome of a living organism. A number of gene editing systems can be used for inserting the nucleotide sequence into a specific genomic locus of the host cell, and some of these systems generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knock-outs, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. In some embodiments, the gene editing systems provided herein for site-specific insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

### ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### TALENs

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39: e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

### Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29 (18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29 (18): 3757-3774. Members of the NHN family are defined by motifs containing two pairs of conserved histidines surrounded by asparagine residues. See Chevalier et al., Nucleic Acids Res. (2001) 29 (18): 3757-3774.

Because the chance of identifying a natural meganuclease for a particular target DNA sequence is low due to the high specificity requirement, various methods, including mutagenesis and high throughput screening, have been used to create meganuclease variants that recognize unique sequences. Strategies for engineering a meganuclease with altered DNA binding specificity, for example, to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363 (2): 283-294.

Like ZFNs and TALENs, Meganucleases can create DSBs in the genomic DNA, which can create a frameshift mutation if improperly repaired, for example, via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### Transposases

Transposases are enzymes that bind to the end of a transposon and catalyze its movement to another part of the genome by a cut and paste mechanism or a replicative transposition mechanism. By linking transposases to other systems, such as the CRISPR/Cas system, new gene editing tools can be developed to enable site-specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems, which use a complex of multiple Cas proteins to degrade nucleic acids; and class 2 systems, which use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096): 816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells, including human cells. In its use in gene editing applications, artificially designed, synthetic gRNAs have replaced the original crRNA:tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing.

### Additional Modifications

In some embodiments, in addition to introducing the nucleic acids comprising a nucleotide sequence encoding a TCR or a fragment thereof according to various embodiments of the present technology into a host cell and integrating the nucleic acids into the genome of the host cell, the host cell may be further modified for use in adoptive therapy.

In some embodiments, the host cells may be further modified to reduce or eliminate expression of the endogenous TCR, so that the host cell would only express the exogenous TCR or fragment thereof encoded by the transgene. Disruption of expression of the endogenous TCR may be accomplished by knocking out, knocking down, or otherwise modifying one or more endogenous TCR genes (e.g., TRAC, TRBC1, and/or TRBC2) using a gene editing system as described. As used herein, “knock out” includes deleting all or a portion of the target nucleotide sequence in a way that interferes with the function of the target gene. For example, a knockout can be achieved by altering a target nucleotide sequence by inducing an indel in a functional domain of the target nucleotide sequence (e.g., a DNA binding domain) or where base editing and prime editing can be used to change single nucleic acid bases to an alternate base in order to alter the genome sequence. “Knock down” refers to genetic modifications that result in reduced expression of the edited gene. As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A gene editing system, for example, the CRISPR/Cas system, of the present disclosure can be used to induce an indel of any length in a target polynucleotide sequence. In some embodiments, a transgene (i.e., a nucleic acid encoding a TCR or a fragment thereof according to various embodiments of the present technology) may be inserted into an endogenous TCR gene locus (knocking in) to disrupt expression of that gene using a gene editing system as described, for example, the CRISPR/Cas system.

In some embodiments, the one or more endogenous TCR genes to be knocked out, knocked down, or otherwise modified include, but are not limited to, TRAC, TRBC1, and/or TRBC2. TCRs recognize foreign antigens processed as small peptides and bound to MHC molecules at the surface of APCs. Each TCR is a dimer consisting of one alpha and one beta chain (most common) or one delta and one gamma chain. The genes encoding the TCR alpha chain are clustered on chromosome 14. The TCR alpha chain is formed when one of at least 70 variable (V) genes, which encode the N-terminal antigen recognition domain, rearranges to 1 of 61 joining (J) gene segments to create a functional variable region that is transcribed and spliced to a constant region gene segment encoding the C-terminal portion of the molecule. The beta chain, on the other hand, is generated by recombination of the V, D (diversity), and J segment genes. The TRAC gene encodes the TCR alpha chain constant region. The human TRAC gene resides on chromosome 14 at 22,547,506-22,552,156, forward strand. The TRAC genomic sequence is set forth in Ensembl ID ENSG00000277734. The TRBC gene encodes the TCR beta chain constant region. TRBC1 and TRBC2 are analogs of the same gene, and T cells mutually exclusively express either TRBC1 or TRBC2. The human TRBC1 gene resides on chromosome 7 at 142,791,694-142,793,368, forward strand, and its genomic sequence is set forth in Ensembl ID ENSG00000211751. The human TRBC2 gene resides on chromosome 7 at 142,801,041-142,802,748, forward strand, and its genomic sequence is set forth in Ensembl ID ENSG00000211772. In some embodiments, the knocking out, knocking down, or otherwise modifying one or more endogenous TCR genes (e.g., TRAC, TRBC1, and/or TRBC2) occurs in both alleles of the genomic locus. In some embodiments, the knocking out, knocking down, or otherwise modifying one or more endogenous TCR genes (e.g., TRAC, TRBC1, and/or TRBC2) occurs in one allele of the genomic locus.

In some embodiments, the host cells may be further modified to reduce the immunogenicity of host cells, in order to reduce potential graft-versus-host risks after infusion into a recipient or risks of being eliminated by the recipient's innate immune system. In cell therapy, when the host cells are allogeneic (i.e., derived from a person other than the recipient), additional modifications are needed to reduce potential graft-versus-host risks after infusion into the recipient or risks of being eliminated by the recipient's innate immune system. In some embodiments, the additional modifications comprise reducing or eliminating the expression of MHC class I and/or II molecules (or HLA class I and/or II molecules) in the host cells. In some embodiments, the allogeneic host cells may be modified by knocking out, knocking down, or otherwise modifying one or more of the HLA loci, such as HLA-A, HLA-B, and/or HLA-C, for example, by using the CRISPR/Cas system as described. By modulating (e.g., reducing or deleting) expression of any of the HLA genes, the cells can be rendered hypoimmunogenic and have a reduced ability to induce an immune response in a recipient subject. In some embodiments, the host cells may be further modified to protect them from natural killer (NK) cell-mediated killing after infusion into a recipient, for example, by additionally expressing one or more NK inhibitory ligands, and/or by expressing nonclassical HLA-E and/or HLA-G. See Biernacki et al., Cancer J. (2019) 25 (3): 179-190; Torikai et al., Blood (2013) 122 (8): 1341-1349, the entire contents of each of which are incorporated by reference herein.

In some embodiments, the host cells may be further modified to express one or more cytokines, growth factors, and other factors that can be used to manipulate the recipient's immune response towards anticancer activity and/or support engraftment of the host cell into the recipient. Cytokines useful for promoting immune anticancer or antitumor response include, for example, IFN-α, IL-2, IL-3, IL-4, IL-7, IL-9, IL-10, IL-12, IL-13, IL-15, IL-16, IL-17, IL-18, IL-21, IL-24, and GM-CSF.

In some embodiments, the host cells may be further modified to express one or more safety switches to induce death or apoptosis of the modified host cells, for example, if the cells grow and divide in an undesired manner or cause excessive toxicity to the recipient. Thus, the use of safety switches enables one to conditionally eliminate aberrant cells in vivo and can be a critical step for the application of cell therapies in the clinic. Safety switches and their uses thereof are described in, for example, Düzgüneş, Origins of Suicide Gene Therapy (2019); Düzgüneş (eds), Suicide Gene Therapy. Methods in Molecular Biology, vol. 1895 (Humana Press, New York, NY) (for herpes simplex virus thymidine kinase (HSVtk), cytosine deaminase (CyD), nitroreductase (NTR), purine nucleoside phosphorylase (PNP), and horseradish peroxidase); Zhou and Brenner, Exp Hematol 44 (11): 1013-1019 (2016) (for inducible caspase 9 (iCasp9)); Wang et al., Blood 18 (5): 1255-1263 (2001) (for huEGFR); U.S. Patent Application Publication No. 20180002397 (for HER1); and Philip et al., Blood 124 (8): 1277-1287 (2014) (for RQR8). In some embodiments, the safety switch can cause cell death in a controlled manner, for example, in the presence of a drug or prodrug or upon activation by a selective exogenous compound. In some embodiments, the safety switch comprises a “suicide gene” or “suicide switch”. The suicide gene can cause the death of the cells should they grow and divide in an undesired manner. The suicide gene may encode a protein that results in cell killing only when activated by a specific compound, for example, an enzyme that selectively converts a nontoxic compound into highly toxic metabolites. In some embodiments, the safety switch may be a membrane-expressed protein which allows for cell depletion after administration of a specific antibody to that protein. In some embodiments, the safety switch is selected from the group consisting of HSVtk, CyD, NTR, PNP, horseradish peroxidase, iCasp9, rapamycin-activated caspase 9 (rapaCasp9), CCR4, CD16, CD19, CD20, CD30, EGFR, GD2, HER1, HER2, MUC1, PSMA, and RQR8.

### Host Cells and Compositions Thereof

In some aspects, provided are host cells, such as T cells, that contain the nucleic acids comprising a nucleotide sequence encoding a TCR or a fragment thereof, and/or express the exogenous TCR or fragment thereof, according to various embodiments of the present technology.

In some embodiments, nucleic acids of the present technology are used in the form of a vector or a virus as described to transfect/transduce a host cell, such as a T cell, for use in adoptive cell therapy to target SF3B1^{K700E}.

In some embodiments, the host cell is a T cell. In some embodiments, the T cell is a CD4+ T cell, a CD8+ T cell, a CD4-CD8-double negative T cell, a naïve T cell (CD62L+, CCR-7+, CD45RA+, CD25−, CD45RO−), a central memory T cell (CD62L+, CCR-7+, CD45RA−, CD45RO+, CD25+, CD127+), an effector memory T cell (CD62L−, CCR-7−, CD45RA−, CD45RO+, CD25−, CD127+), a stem cell memory T cell (CD62L+, CCR-7+, CD45RA+, CD45RO+), or any combination thereof. In some embodiments, the T cell is a cytotoxic T cell, or a CD8+ T cell.

In some embodiments, the T cells are autologous (i.e., obtained from the subject who will receive the T cells after modification). In some embodiments, the T cells are allogeneic (i.e., obtained from someone other than the subject who will receive the T cells after modification). In either of these embodiments, the T cells can be primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, for example, in the case of allogeneic T cells, the T cells can be derived or differentiated from pluripotent stem cells (PSCs), such as embryonic stem cells (ESCs) or induced pluripotent cells (iPSCs).

In some embodiments, an exogenous TCR or a fragment thereof specific to SF3B1^{K700E}, an epitope of SF3B1^{K700E}, and/or an SF3B1^{K700E} epitope/HLA complex according to various embodiments disclosed therein is expressed by a host cell, such as a CD8+ T cell, and the host cell recognizes and initiates an immune response to a target cell expressing SF3B1^{K700E} or an epitope thereof. As explained in greater detail below, the target cell includes cancer cells, such as hematologic cancer cells.

In some aspects, provided are pharmaceutical compositions comprising a host cell according to various embodiments of the present technology.

In some embodiments, the host cell (e.g., a T cell expressing an exogenous TCR of the present technology) may be present in the pharmaceutical composition in an amount greater than about 102/ml, for example, up to about 103/ml, up to about 10⁴/ml, up to about 10⁵/ml, up to about 10⁶/ml, up to about 10⁷/ml, up to about 10⁸/ml, up to about 10⁹/ml, or about 10¹⁰/ml or more.

In some embodiments, the pharmaceutical composition may have various formulations, for example, injectable formulations, lyophilized formulations, liquid formulations, oral formulations, etc., depending on the suitable routes of administration. In some embodiments, the pharmaceutical compositions may have various formulations for injection and/or infusion. Non-limiting examples of formulations for injection and/or infusion include intravenous injection, intraperitoneal injection, intertumoral injection, bone marrow injection, lymph node injection, subcutaneous injection, and cerebrospinal fluid injection.

In some embodiments, the pharmaceutical compositions may be co-formulated in the same dosage unit or can be individually formulated in separate dosage units. The terms “dose unit” and “dosage unit” herein refer to a portion of a pharmaceutical composition that contains an amount of a therapeutic agent, such as a host cell, suitable for a single administration to provide a therapeutic effect. Such dosage units may be administered one to a plurality (i.e., 1 to about 10, 1 to 8, 1 to 6, 1 to 4, or 1 to 2) of times per day, or as many times as needed to elicit a therapeutic response.

In some embodiments, the pharmaceutical compositions may further comprise one or more cytokines, growth factors, and other factors that can be used to manipulate the recipient's immune response towards anticancer activity and/or support engraftment of the host cell into the recipient. Cytokines useful for promoting immune anticancer or antitumor response include, for example, IFN-α, IL-2, IL-3, IL-4, IL-7, IL-9, IL-10, IL-12, IL-13, IL-15, IL-16, IL-17, IL-18, IL-21, IL-24, and GM-CSF.

In some embodiments, the pharmaceutical compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like, and combinations thereof. In some embodiments, compositions comprising host cells, as disclosed herein, further comprise a suitable infusion media.

### Methods of Treatment

In some aspects, provided are methods for treating a subject in need thereof having an SF3B1^{K700E}-positive cancer. The methods include administering a therapeutically effective amount of a host cell (e.g., a T cell), or a pharmaceutical composition containing the same, that specifically recognizes SF3B1^{K700E}, an epitope of SF3B1^{K700E}, and/or an SF3B1^{K700E} epitope/HLA complex, to the subject according to various embodiments of the present technology. In some embodiments, the cancer cell expresses SF3B1^{K700E}, or an epitope thereof. In some embodiments, SF3B1^{K700E}, or an epitope thereof, is presented on the surface of the cancer cell via a MHC protein, for example, an HLA complex. In some embodiments, SF3B1^{K700E}, or an epitope thereof, is presented on the surface of the cancer cell via HLA-B*40:01.

In some embodiments, the cancer is a hematologic malignancy. Non-limiting examples of hematologic malignancies include myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

In some embodiments, the host cell, or the pharmaceutical composition containing the same, according to the present technology may be administered in a manner appropriate to the disease, condition, or disorder to be treated as determined by persons skilled in the medical art. In any of the above embodiments, a host cell, such as a T cell expressing an exogenous TCR as described herein, is administered intravenously, intraperitoneally, intertumorally, intratumorally, into the bone marrow, into a lymph node, or into the cerebrospinal fluid so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In some embodiments, the host cells (e.g., a T cell expressing an exogenous TCR of the present technology) are typically administered to the subject in an amount of greater than about 10², for example, up to about 10³, up to about 10⁴, up to about 10⁵, up to about 10⁶, up to about 10⁷, up to about 10⁸, up to about 10⁹, or about 10¹⁰ or more per dose.

In some embodiments, the methods comprise administering to the subject host cell, or a pharmaceutical composition containing the same, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the host cell, or the pharmaceutical composition containing the same, can be administered every day, every other day, every third day, weekly, biweekly (i.e., every other week), every third week, monthly, every other month, or every third month. In some embodiments, the host cell, or the pharmaceutical composition containing the same, can be administered continuously or intermittently, for example, in one or more cycles. In those embodiments, within each cycle, the host cell, or the pharmaceutical composition containing the same, can be administered at various lengths and/or frequencies as described above.

In some embodiments, the host cell, or the pharmaceutical composition containing the same, may be administered over a pre-determined time period. Alternatively, the host cell, or the pharmaceutical composition containing the same, may be administered until a particular therapeutic benchmark is reached. In some embodiments, the methods provided herein include a step of evaluating one or more therapeutic benchmarks in a biological sample, such as, but not limited to, the level of a cancer biomarker, to determine whether to continue administration of the host cell, or the pharmaceutical composition containing the same.

In some embodiments, the method further entails administering one or more other cancer therapies, such as surgery, immunotherapy, radiotherapy, chemotherapy, and/or transplantation, to the subject sequentially or simultaneously. For treatment of hematologic malignancies, the transplantation can be HCT, also referred to as bone marrow transplantation or stem cell transplantation. Depending on the source of the transplanted hematopoietic cells, HCT can be autologous (i.e., the healthy cells are collected from the subject) or allogenic (the healthy cells are collected from a donor). In some embodiments, the transplantation (e.g., HCT) comprises administering to the subject a conditioning regimen prior to the transplantation, which is designed to provide adequate immunosuppression to prevent rejection of the transplanted cells (e.g., in the case of allogenic HCT) and to eradicate the cancer cells in the subject before the transplantation. A conditioning regimen may include chemotherapy, radiation, and monoclonal antibody therapy. For example, during an HCT, a subject may be given high doses of chemotherapy and/or radiation to kill cancer cells. Healthy hematopoietic cells collected from the subject prior to the chemotherapy and/or radiation, or from a donor, can be transplanted to reestablish the blood cell production process in the bone marrow. In some embodiments, the conditioning regimen may comprise administering one or more chemotherapy agents to the subject, for example, fludarabine (Flu), or fludarabine and cyclophosphamide (Flu/Cy).

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent, a chemotherapy agent, and a biologic agent. In some embodiments, the subject was administered the one or more additional therapeutic agents before administration of the host cell, or the pharmaceutical composition containing the same. In some embodiments, the subject is co-administered the one or more additional therapeutic agents and the host cell, or the pharmaceutical composition containing the same. In some embodiments, the subject was administered the one or more additional therapeutic agents after administration of the host cell, or the pharmaceutical composition containing the same.

As one of ordinary skill in the art would understand, the one or more additional therapeutic agents and the host cell, or the pharmaceutical composition containing the same, can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

### EXAMPLES

### Example 1: Identification of Putative Spliceosome Neoantigens

The objective of this example was to identify spliceosome neoantigens that are specific to MDS and sAML cancer cells and thus may serve as potential targets for immunotherapies. We first investigated whether aberrant amino acid sequences resulting from recurrent spliceosome gene mutations had potential to produce candidate MDS and sAML epitopes. Although neoantigens could theoretically be created from any protein-coding mutation, aberrant peptides will not be processed from the protein product of every protein-coding mutation, not every aberrant peptide that is processed will be presented all or even some HLA molecules, and not every aberrant peptide-HLA complex will be immunogenic. Predicting which mutations are likely to lead to immunogenic, naturally processed and presented epitopes streamlines the process of neoantigen discovery and immunotherapy development by allowing investigators to focus on epitopes that are most likely to be biologically relevant.

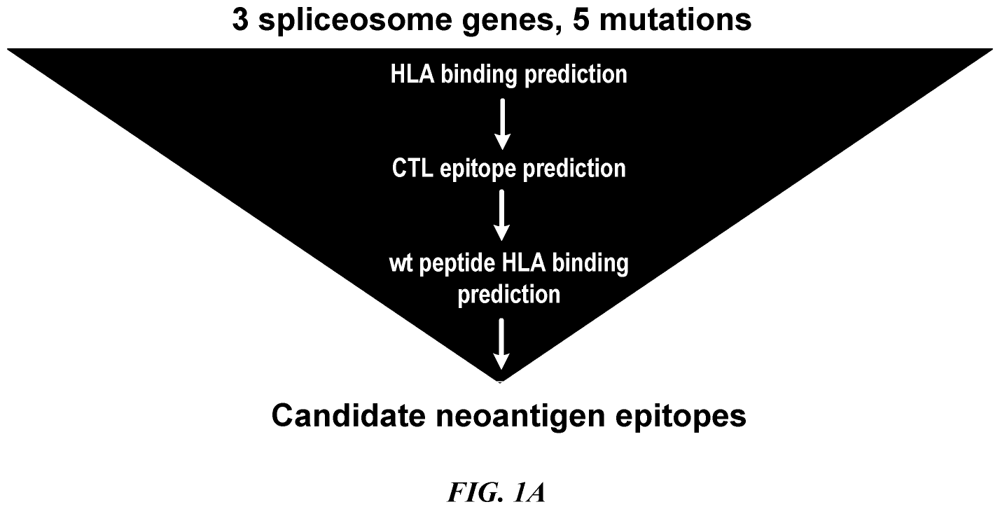

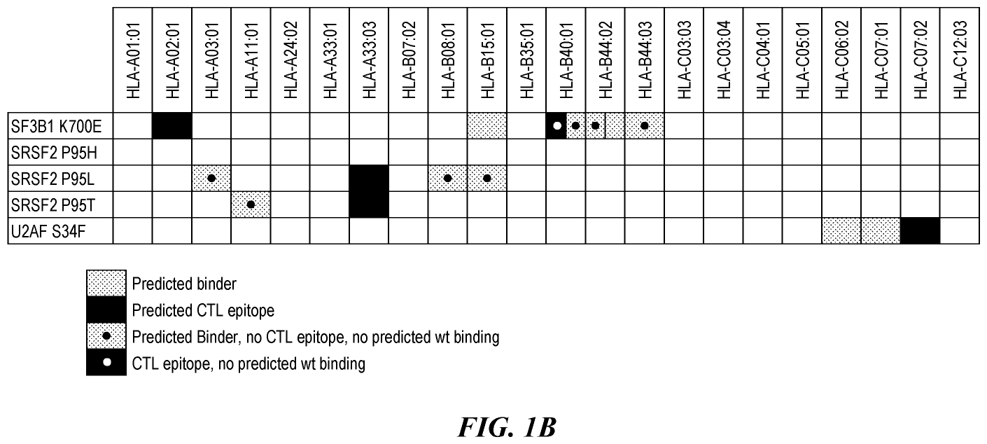

Although most existing algorithms do not reliably predict immunogenicity, there are generally four features needed for epitopes to elicit T cell responses: (1) strong peptide binding to HLA; (2) cleavage of the peptide from the parent protein; (3) loading of the peptide onto HLA; and (4) absence of tolerance to the epitope, for example, due to failure of the wild-type peptide to bind the restricting HLA. We performed a multi-step in silico analysis to screen for candidate spliceosome neoantigen epitopes (FIG. 1A). We first used the HLA binding prediction algorithms netMHCpan 4.1 and the Immune Epitope DataBase (IEDB) consensus algorithms Artificial Neural Network (ANN) and Stabilized Matrix Method (SMM) to identify spliceosome mutation-derived peptides with a high probability of binding strongly to any of 22 HLA class I molecules, including 6 HLA-A, 7 HLA-B, and 8 HLA-C alleles. We evaluated mutations that were expected to occur in >3% in patients with spliceosome mutated MDS or sAML based on published frequencies. Epitopes were considered candidates if they were predicted by at least two metrics: binding affinities of less than 250 nM for IEDB ANN or SMM artificial or for netMHCpan binding affinity prediction; and percent rank less than one for mass spectrometry-based predictions in netMHCpan. Sixteen candidate epitopes were predicted from mutations in SF3B1, SRSF2, and U2AF1 (FIG. 1A, Table 8). We next assessed which of these epitopes were likely to be processed from the parent protein and loaded onto HLA using the algorithm netCTLpan, which incorporates predictions of proteasomal cleavage and peptide transport. Analysis of a small set of epitopes known to be immunogenic, processed, and presented (Table 9) suggested that setting a threshold rank <1% captured the majority of processed and presented epitopes. Using these criteria, we identified five peptides with binding to six different HLA molecules that were predicted to yield 7 CTL epitopes (FIG. 1A, Table 8). Finally, we evaluated the probability of the equivalent wild-type peptides binding to the predicted HLA molecules for all candidate neoantigen epitopes, reasoning that tolerance to a candidate neoantigen epitope would be likely if the wild-type peptide were presented. Wild-type peptides were predicted not to bind for 9 of the candidate epitopes (FIG. 1B, Table 8), three of which were also predicted CTL epitopes.

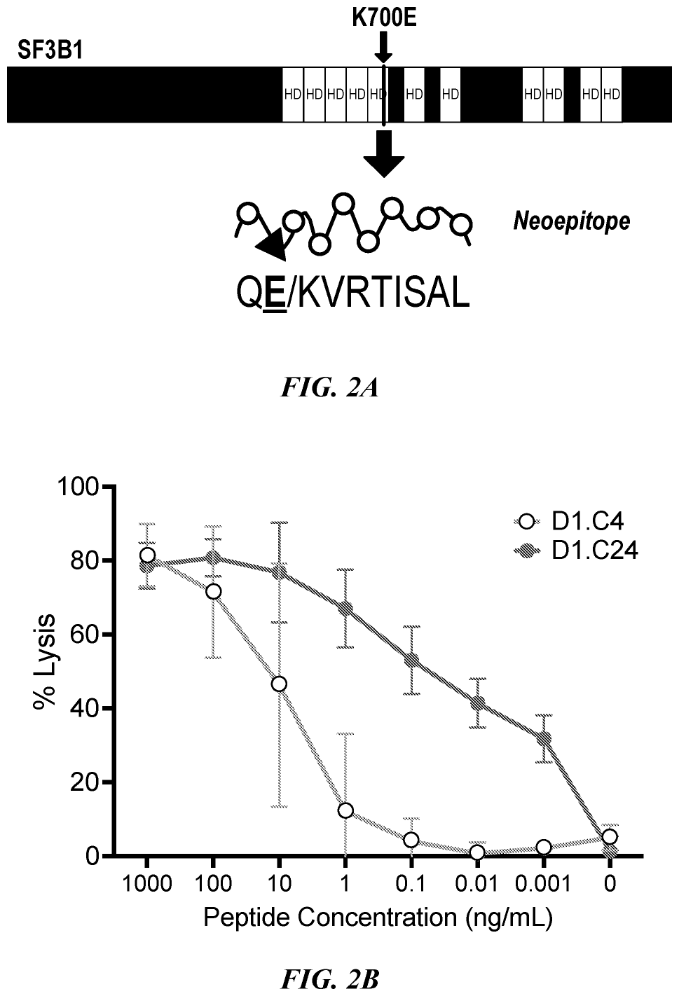

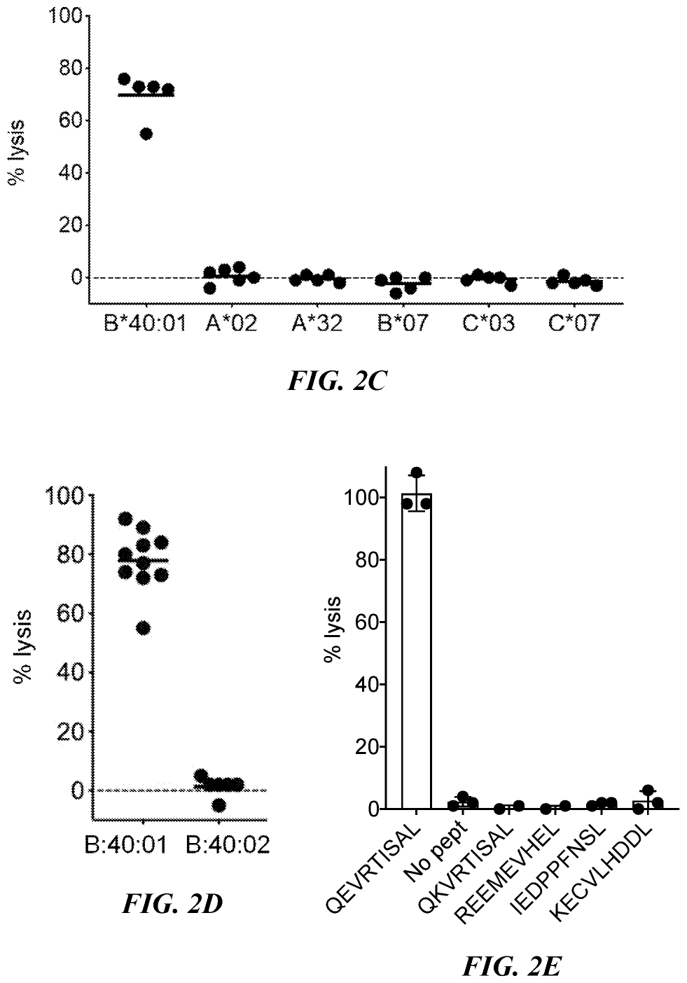

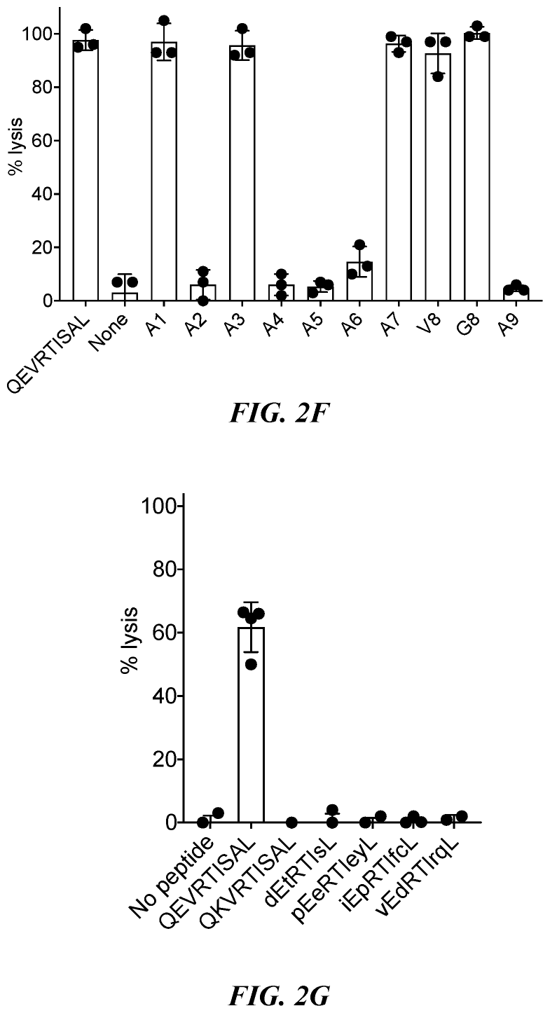

Three HLA molecules, HLA-B*40:01, -B*44:02, and -B*44:03, were predicted to present the same SF3B1^{K700E} peptide. Since the predicted affinity for HLA-B*40:01 was over 20-fold stronger than for HLA-B*44:02 or -B*44:03 (14 nM versus 278 nM or 250 nM, respectively; Table 8), we focused on HLA-B*40:01 as the most promising predicted HLA restriction. HLA binding and CTL epitope prediction algorithms identify peptides that are likely epitopes, but predictions require validation. For example, one candidate epitope, QEVRTISAL (SEQ ID NO: 1) (FIG. 2A), had strong predicted binding to HLA-B*40:01 by both binding affinity (14 nM) and rank (0.034) (Table 5). The corresponding peptide from the wild-type (WT) sequence, QKVRTISAL (SEQ ID NO: 2) (FIG. 2A), did not have strong predicted binding to HLA-B*40:01 (Table 5).

TABLE 5
Predicted binding of SF3B1^{K700E} epitope and WT
SF3B1 epitope to HLA-B*40:01

| | | HLA-B*40:01 | |
|---|---|---|---|
| Parent | Peptide | nM | Rank |
| SF3B1^{K700E} | QEVRTISAL | 14.14 | 0.034 |
| | (SEQ ID NO: 1) | | |
| SF3B1 WT | QKVRTISAL | 10334.24 | 2.676 |
| | (SEQ ID NO: 2) | | |

This represents the first study to describe neoantigens created directly from the protein products of recurrent spliceosome mutations, including SF3B1. These neoantigens are distinct from those that have been identified for de novo AML resulting from a recurrent NPM1 mutation or a leukemia-initiating CBFB-MYH11 gene fusion, neither of which are found in MDS. Neoantigens resulting from mutations in TP53 found in cancers broadly also should occur in some MDS and sAML. While TP53 mutation-derived neoantigens are potential T cell therapy targets for MDS and would benefit high-risk patients particularly, these are typically subclonal rather than clonal mutations. Consequently, therapies targeting TP53-derived neoantigens would likely be subject to leukemia escape and not curative. In contrast, therapies targeting neoantigens resulting from spliceosome mutations should be able to eradicate the founding neoplastic clone.

Isolation of T cells specific for candidate neoantigens enables confirmation that the antigen is adequately processed and presented on the surface of malignant cells to induce T cell killing and thus represents a bona fide cancer neoantigen. Thus, we next directly tested the immunogenicity of the most promising candidate, the SF3B1^{K700E} neoantigen, by primary in vitro stimulation of CD8+ T cells from HLA-typed healthy donors with each candidate MDS/sAML neoantigen.

### Example 2: SF3B1^{K700E} Neoepitope is Immunogenic and Elicits High-Avidity T Cell Responses

The objective of this example was to identify peptide epitopes from SF3B1^{K700E} mutation that can be used for the development of T cell-based immunotherapy. We first examined SF3B1^{K700E}/B*40:01. To confirm that the predicted peptide did bind to HLA-B*40:01 and could stimulate T cell responses, the immunogenicity of the candidate SF3B1^{K700E} epitope was tested in vitro by stimulating CD8+ T cells from an HLA-B*40:01⁺ healthy donor with autologous monocyte-derived dendritic cells pulsed with a pool of peptide epitopes including controls and the SF3B1^{K700E} peptide (i.e., QEVRTISAL (SEQ ID NO: 1)). After 12-13 days of in vitro stimulation and culture, specific lysis of peptide-pulsed autologous LCL by the stimulated T cells was evaluated in 51Cr-release cytotoxicity assay (CRA). T cells that specifically lysed peptide-pulsed targets were cloned by limiting dilution, expanded, and tested for recognition of the SF3B1^{K700E} peptide. Stimulation of CD8+ T cells from HLA-B*40:01+ donor elicited responses to the QEVRTISAL (SEQ ID NO: 1) peptide from SF3B1^{K700E} that was predicted to bind HLA-B*40:01. Two QEVRTISAL (SEQ ID NO: 1)-specific clones were isolated and cloned by limiting dilution. Clone 24 (D1.C24) demonstrated high functional avidity in CRA with half-maximal lysis around 0.1 ng/ml (FIG. 2B) and recognized only QEVRTISAL (SEQ ID NO: 1) peptide-pulsed LCL bearing HLA-B*40:01 but not LCL with other HLA shared by the original T cell donor, confirming HLA-B*40:01 restriction (FIG. 2C). HLA-B*40:02 is less frequent but structurally similar to HLA-B*40:01 and may present the same peptides. However, although the predicted affinity of QEVRTISAL (SEQ ID NO: 1) for HLA-B*40:02 by netMHCpan 4.1 was 25 nM, clone 24 did not cross-recognize the peptide presented on HLA-B*40:02 (FIG. 2D). To confirm that high-avidity clone was truly specific for QEVRTISAL (SEQ ID NO: 1), we tested it against known immunogenic, HLA-B*40:01-presented epitopes, and found that it lysed only autologous LCL pulsed with QEVRTISAL (SEQ ID NO: 1) peptide, not those pulsed with wild-type QKVRTISAL (SEQ ID NO: 2) peptide or any of three known immunogenic, HLA-B*40:01 presented peptides (FIG. 2E).

We next assessed the high-avidity QEVRTISAL (SEQ ID NO: 1)-specific clone for cross-reactivity and the potential for off-target recognition of non-SF3B1^{K700E} peptides. Alanine scanning demonstrated that residues at positions 2 and 9, known anchor residues for HLA-B*40:01-binding, and positions 4, 5, and 6 were required for clone 24 to recognize QEVRTISAL (SEQ ID NO: 1) (FIG. 2F). To assess potential cross-recognition of other similar human peptides, we performed an insilico search for proteins containing the xExRTIxxL motif using the ScanProsite tool. Four wild-type human proteins with the motif were identified: alkylglycerone-phosphate synthase (AGPS), mitochondrial trifunctional enzyme subunit alpha (HADHA), Gem-associated protein 5 (GEMIN5), and spermatogenesis-associated protein 1 (SPATA1) (Table 6). However, none of the peptides with the xExRTIxxL motif were recognized by clone 24 (FIG. 2G), indicating that the clone was highly specific for QEVRTISAL (SEQ ID NO: 1).

TABLE 6
Binding prediction for peptides sharing xExRTIxxL motif

| Peptide | Gene ID | EL_Rank | IC50 (nM) |
|---|---|---|---|
| dEtRTIisL | AGPS | 0.2444 | 346 |
| pEeRTIeyL | HADHA | 1.2886 | 4847 |
| iEpRTIfcL | GEMIN5 | 0.6814 | 479 |
| vEdRTIrqL | SPATA1 | 0.0488 | 83 |

### Example 3: SF3B1^{K700E} Neoepitope is a Bona Fide MDS and sAML Neoantigen

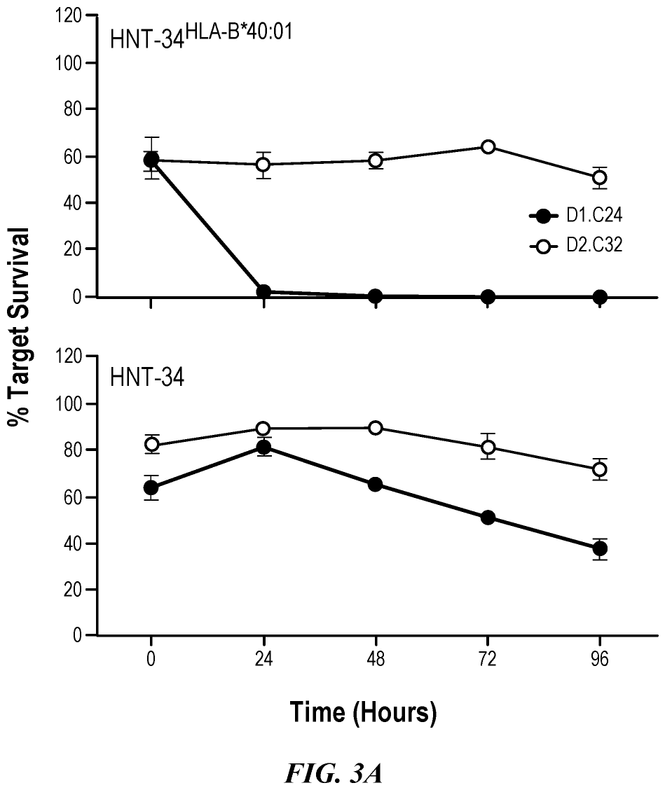

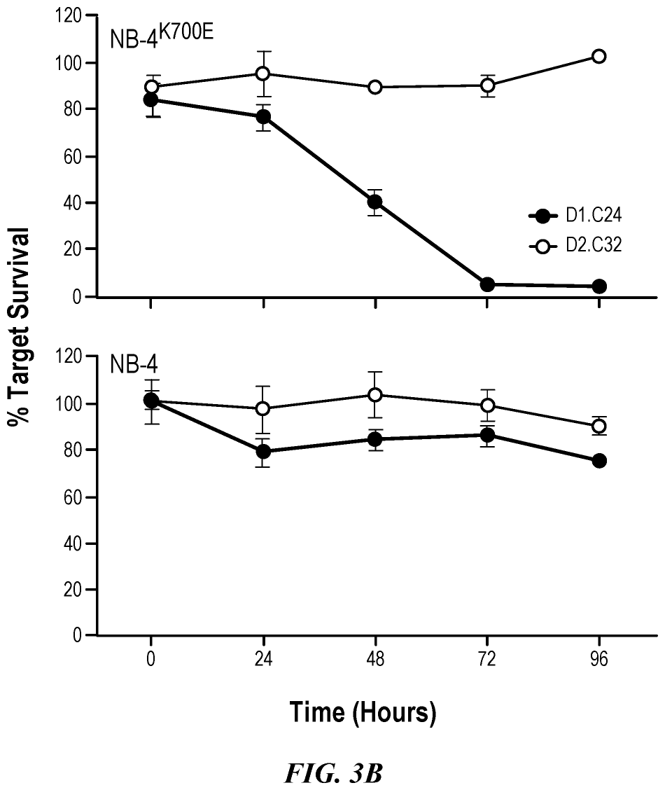

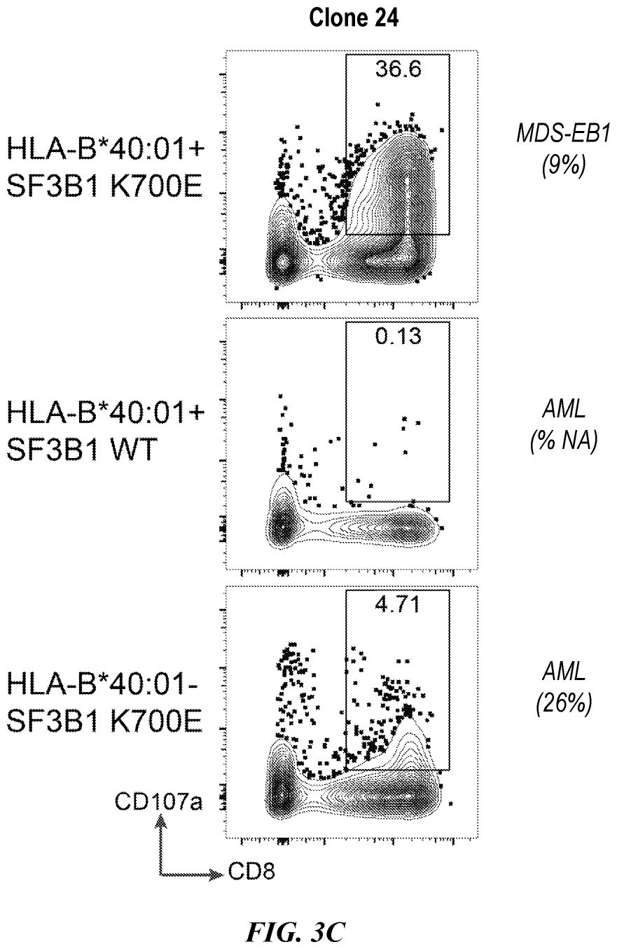

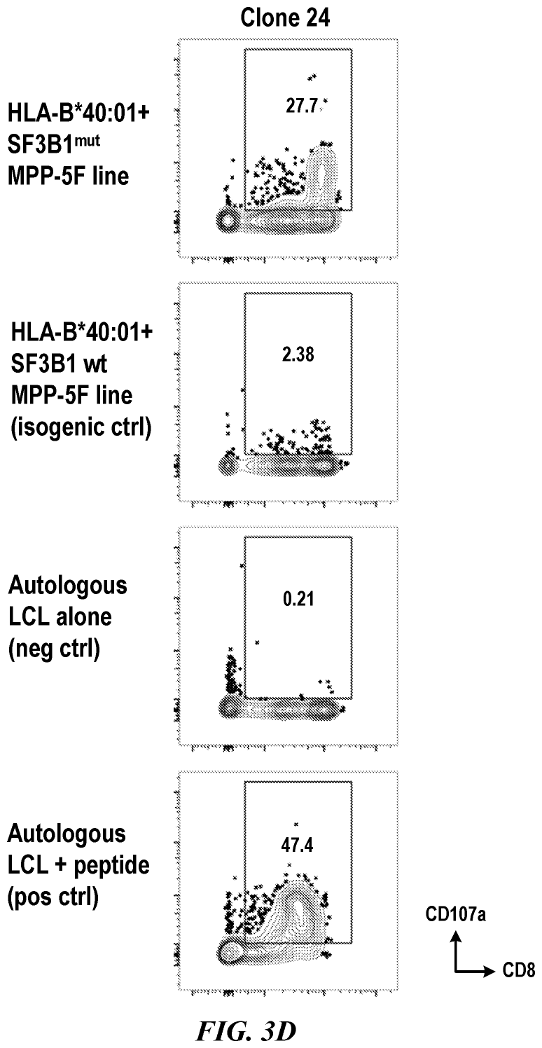

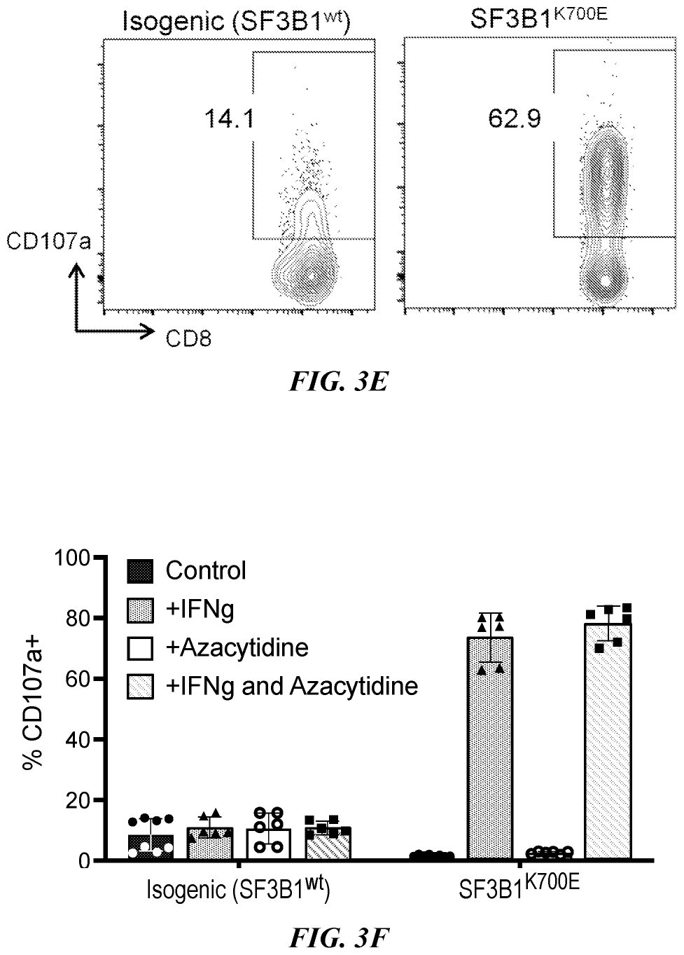

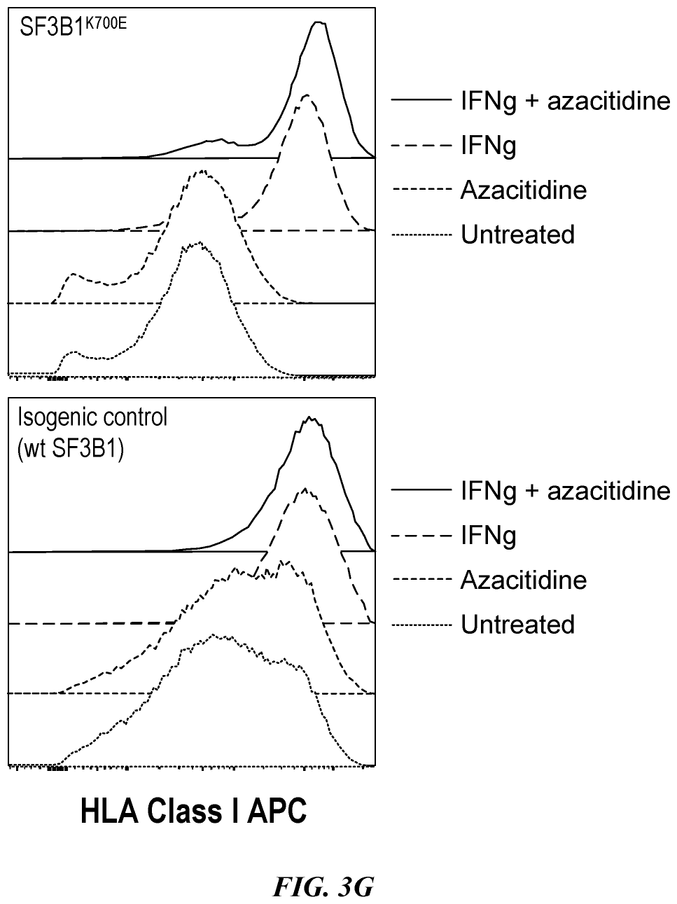

We then investigated whether the QEVRTISAL (SEQ ID NO: 1) epitope was naturally presented on neoplastic myeloid cells, first utilizing cell lines. To create target cells that were genotypically positive for both SF3B1^{K700E} and HLA-B*40:01, we transduced the naturally SF3B1^{K700E}-positive sAML cell line HNT-34 to express HLA-B*40:01. In a flow cytometry-based cytotoxicity assay, clone 24 effectively eliminated HLA-B*40:01-transduced HNT-34 cells by 24 hours, while HNT-34/B*40:01-transduced cells cultured with a control clone specific for an irrelevant neoantigen showed approximately 60% survival (FIG. 3A). To validate recognition of endogenous antigen in a second cell line model, we transduced a minigene encoding SF3B1^{K700E} into the naturally HLA-B*40:01-positive myeloid cell line NB-4. As with the HNT-34 cells, clone 24 eliminated the SF3B1^{K700E} minigene-transduced NB-4 in the cytotoxicity assay, but the control clone had minimal effect (FIG. 3B).

To determine whether the QEVRTISAL (SEQ ID NO: 1) epitope was naturally processed from SF3B1^{K700E} protein and presented by endogenous antigen-presenting machinery on primary malignant myeloid cells, high-avidity SF3B1^{K700E}-specific clones were co-cultured with hematopoietic cells from patients with active MDS or AML, and then assessed for antigen recognition in a CD107a degranulation assay. Clone 24 showed specific degranulation in response to primary SF3B1^{K700E} HLA-B*40:01-positive samples compared to samples lacking either the mutation or restricting HLA (FIG. 3C), indicating that the SF3B1^{K700E} epitope QEVRTISAL (SEQ ID NO: 1) is naturally processed and presented on malignant myeloid cells, and that SF3B1^{K700E}/HLA-B*40:01-specific CD8+ T cells recognize the endogenous antigen. These results indicate that the SF3B1^{K700E} epitope is naturally processed and presented on primary neoplastic hematopoietic cells and is thus a bona fide MDS/sAML neoantigen.

### Example 4: Hematopoietic Progenitor Lines Generated from SF3B1K^{700E}-Positive Primary MDS Cells Present Antigen

One challenge in developing TCR-T immunotherapies for MDS is acquiring adequate numbers of primary cells from patient samples. Cytopenias are a hallmark of MDS, and peripheral blood samples consequently may have a paucity of cells with which to perform assays. Induced pluripotent stem cells (iPSC) can be differentiated into multipotent hematopoietic progenitor cell lines (MPP-5F) that recapitulate the genotype and phenotype of primary patient MDS cells. We thus evaluated whether SF3B1^{K700E}-specific clones could recognize SF3B1^{K700E} iPSC-derived hematopoietic cells. We reprogrammed hematopoietic stem/progenitor cells (HSPC) from SF3B1^{K700E} MDS patient bone marrow and established two iPSC lines: one with SF3B1^{K700E} mutation and an isogenic control with wild-type SF3B1. Multipotent hematopoietic progenitor lines (MPP-5F) were then generated from iPSC lines by doxycycline-dependent expression of five HSPC transcription factors. In a CD107a degranulation assay, high-avidity clone 24 showed specific degranulation in response to SF3B1^{K700E} MPP-5F but not to the isogenic control (FIGS. 3D-3F), indicating that the iPSC-derived MPP-5F line recapitulated presentation of the neoantigen. Notably, pre-culture with interferon-gamma (IFNg) was required for clone 24 to recognize the SF3B1^{K700E} MPP-5F line. We determined that this was due to low baseline expression of HLA class I on MPP-5F that was ameliorated by IFNg exposure (FIG. 3G). Thus, iPSC-derived lines might facilitate immunotherapy development for MDS, as they could serve as surrogates for primary MDS cells. Together, these results suggest that the identified SF3B1^{K700E} epitope is a novel neoantigen with promise as an immunotherapy target for MDS and other hematologic malignancies with SF3B1 mutations, including secondary AML, MDS/myeloproliferative neoplasm overlap syndrome, and advanced CLL.

### Example 5: MDS Neoepitope-Specific TCR can be Transferred and Confer Specificity

Immunotherapies utilizing adoptive transfer of T cells engineered to express antigen-specific TCRs can overcome numerical or functional defects in natural T cell immunity against malignant cells. Having discovered the SF3B1^{K700E} neoantigen, we next assessed the feasibility of transferring TCRs specific for this neoantigen as a step towards clinical translation. We first sequenced the TCR alpha and beta chains from the SF3B1^{K700E}/B*40:01-specific T cell clone using a next-generation sequencing platform (Adaptive Biotechnologies). For example, Table 7 provides TCR sequence information from the sequenced SF3B1^{K700E}/HLA-B*40:01-specific CD8+ T cell clone:

TABLE 7
Specificity, V region usage, and amino acid sequence data for the TCR alpha
and beta chains identified by sequencing of the C24 clone

| | | HLA | | | | | |
|---|---|---|---|---|---|---|---|
| Clone | Peptide | restriction | Chain | V gene | IMGT CDR1 | IMGT CDR2 | IMGT CDR3 |
| 24 | QEVRTISAL | B*40:01 | alpha | TCRAV08- | YGATPY (SEQ | YFSGDTLV | CAVAAKDSSYKLIF |
| | (SEQ ID | | | 03 | ID NO: 12) | (SEQ ID | (SEQ ID NO: 14) |
| | NO: 1) | | | | | NO: 13) | |
| | | | beta | TCRBV29- | SQVTM (SEQ | ANQGSEA | CSDRGQVVGETQYF |
| | | | | 01 | ID NO: 6) | (SEQ ID | (SEQ ID NO: 8) |
| | | | | | | NO: 7) | |

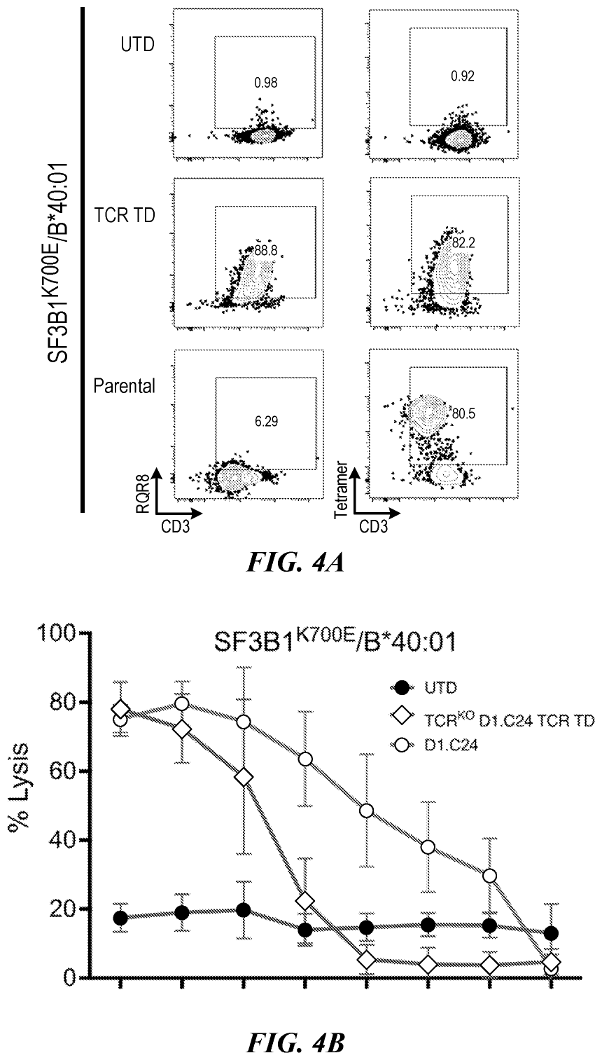

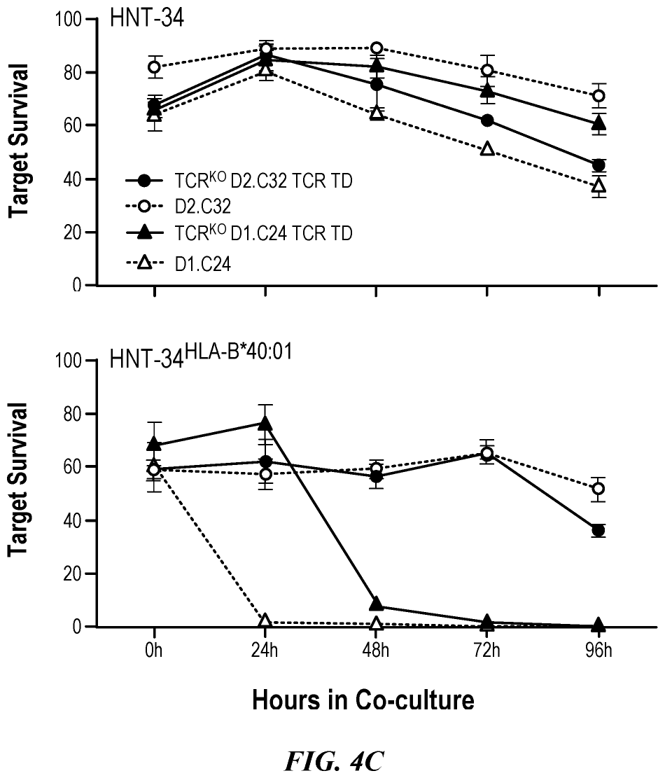

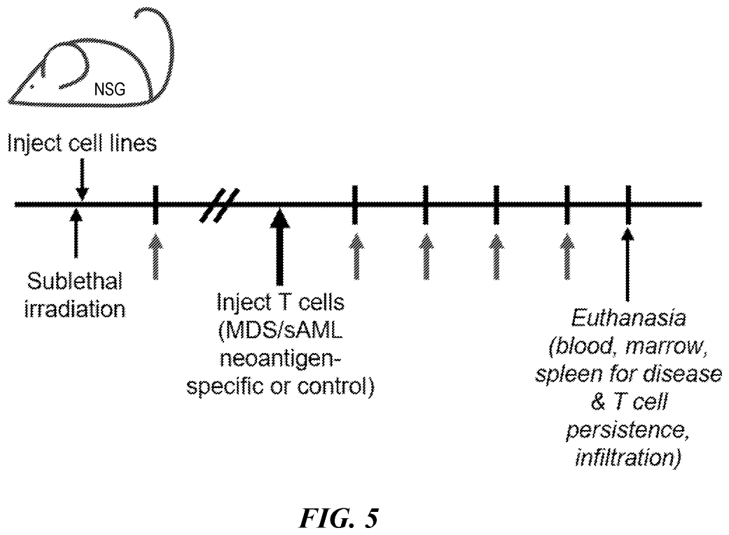

To generate transgenic TCRs, we then paired the TCR alpha and beta chains from each clone in lentiviral vectors (LV). We successfully cloned TCRs specific for the SF3B1^{K700E} neoantigen into LV and transduced healthy donor CD8+ T cells. Primary human CD8+ T cells transduced with the SF3B1^{K700E}/B*40:01-specific TCR, but not untransduced controls, stained with SF3B1^{K700E}/B*40:01 pHLA tetramer along with the RQR8 transduction marker (FIG. 4A). T cells transduced with the SF3B1^{K700E}/B*40:01-specific TCR had lower functional avidity to the parental clone 24 in peptide titration CRA (FIG. 4B) but killed HLA-B*40:01-transduced HNT-34 cell lines bearing both SF3B1^{K700E} and HLA-B*40:01 (FIG. 4C).

### Example 6: Spliceosome Neoantigen-Specific T Cells Control Myeloid Neoplasia In Vivo

We developed a model to evaluate the efficacy of spliceosome neoantigen-specific TCR-T cell in vivo. SF3B1^{K700E}/B*40:01-specific TCR-T cells will be tested in vivo in a cell line-derived xenograft (CDX) model in which immunodeficient NOD scid gamma (NSG) mice are engrafted with the naturally SF3B1^{K700E}-positive HNT-34 cell line transduced to express HLA-B*40:01. Two weeks after intravenous injection of 1×10⁶ HNT-34 cells transduced with HLA-B*40:01 and luciferase, mice will be treated either with vehicle or a single dose of 10×10⁶ CD8+ T cells transduced with the TCR derived from high-avidity clone 24 or with an irrelevant neoantigen-specific control TCR (FIG. 6).

### Example 7: Spliceosome Neoantigen-Specific T Cells do not Damage Hematopoietic Colony Formation

Targeting antigens that are not presented on non-neoplastic hematopoietic cells are crucial for T cell therapies that preserve normal hematopoiesis and are thus suitable for use in all patients with myeloid neoplasms, including those who are HCT-ineligible or have recently undergone HCT. Neoantigens, being absent from normal hematopoietic cells, should be ideal targets for proof-of-concept for hematopoiesis-preserving T cell therapies.

Current strategies include developing neoantigen-directed T cell immunotherapy for MDS that uses ex vivo autologous T cells expanded with peptides based on patient-specific mutations. This approach, however, relies upon the ability to expand functional neoantigen-specific T cell responses, which may be challenging in the immune dysregulated microenvironment associated with MDS. Preliminary data suggests that while manufacturing is feasible and administration is safe, the ex vivo-expanded T cell products are heterogeneous, and their persistence is limited. In contrast, the approach of the present technology uses TCR transfer and enables patients to receive a product with a defined specificity and potency. TCR constructs can be modified further to improve the safety and efficacy of the transduced T cells by including CD8 co-receptors, safety switches, immunomodulatory fusion proteins, and/or cytokine genes. Thus, while TCR-T cell therapy is relatively technically complex compared to ex vivo-expanded T cells, T cell engineering offers a number of advantages over neoantigen-directed T cell lines for MDS and sAML patients. The results here provide key foundational data for developing neoantigen-specific TCR-T cell therapies for myeloid neoplasms.

### Example 8: Materials and Methods

The following experimental materials and methods were used in the above examples.

### Study Approval

Blood samples from healthy volunteer donors and patients with AML were obtained after written informed consent in accordance with the Declaration of Helsinki to participate in research protocols approved by the Institutional Review Board of the Fred Hutchinson Cancer Center (FHCC). Samples from AML patients were obtained through the FHCC/University of Washington Hematopoietic Diseases Repository (protocol #1690). Animal experiments were approved by the Institutional Animal Care and Use Committee of the FHCC (protocol #50941).

### Human Samples and Cell Lines

Blood and bone marrow samples were obtained from healthy volunteer donors and patients with MDS and AML. Mononuclear cells were isolated from non-mobilized apheresis product, whole blood, or bone marrow by Ficoll-Hypaque (Perkin-Elmer) density gradient centrifugation. Peripheral blood mononuclear cells (PBMC) and bone marrow mononuclear cells (BMMC) were cryopreserved in RPMI 1640 supplemented with 20% fetal bovine serum and 10% dimethylsulfoxide (DMSO) in vapor-phase liquid nitrogen in aliquots until use. In some cases, patient AML was expanded by serially transplanting BMMC into immunodeficient humanized MISTRG mice and harvested from secondary recipients prior to use in experiments. Other cell lines were purchased from the American Type Culture Collection (ATCC) or the Deutsche Sammlung von Mikroorganismen und Zellkulturen (DSMZ) and cultured in recommended conditions.

### HLA Binding Prediction Analysis and Peptides

IEDB ANN, IEDB Stabilized Matrix (SMM), and netMHCpan 4.1 were used to predict peptide binding to 22 HLA molecules. Peptides were defined as candidate epitopes if they had predicted IC50<250 nM for any HLA by >1 algorithm. CTL epitope predictions were performed using netCTLpan 1.1 for the same 22 HLA class I molecules. Control and neoantigen epitope peptides were synthesized using standard Fmoc chemistry (Genscript), reconstituted to a stock concentration of 10 mg/mL in DMSO, and stored at −20° C. in aliquots until use.

### Immunogenicity Screening and Identification of Spliceosome Neoantigen-Specific CD8+ T Cells

CD8+ T cells from HLA-typed volunteer PBMC were purified by immunomagnetic bead depletion of CD8-cells (CD8+ T cell isolation kit, Miltenyi Biotec). Autologous dendritic cells (DC) generated from monocytes by a modified fast DC protocol were used as antigen-presenting cells. For each immunogenicity screen, a minimum of 10×10⁶ CD8+ T cells were plated at 3-6×10⁴ T cells per well in 96-well plates, along with autologous mature DC in a T cell: DC ratio of 30:1. A total of up to seven plates were used in each experiment. Prior to co-culture with CD8+ T cells, DC were incubated for 2 hours at 37° C. with putative spliceosome neoantigen epitope and control peptides based on donor HLA type. Each peptide was used at a final concentration of 1 mg/mL in culture medium for the incubation. After incubation with peptides, DCs were then irradiated and washed before co-culturing with T cells. Cultures were supplemented with interleukin (IL)-12 (10 ng/ml) at initiation and IL-15 (10 ng/ml) at day 7. Split-well 51Cr-release cytotoxicity assays (CRA) were performed on day 12-13, using LCL with or without peptide as target cells. A well was considered positive if it exhibited >20% lysis of peptide-pulsed LCL and lysis of peptide-pulsed LCL was >2-fold higher than LCL without peptide. Peptide-specific T cells were cloned by limiting dilution using OKT3, IL-2, and feeder cells, then screened by split-well CRA on day 11-13. A clone was considered positive if it exhibited >20% lysis of peptide-pulsed LCL and lysis of peptide-pulsed LCL was >5-fold higher than lysis of LCL without peptide. Positive clones were expanded using OKT3, IL-2, and feeder cells, and their specificity and functional avidity were evaluated by peptide-HLA (pHLA) tetramer staining and functional assays (e.g., CRA and flow cytometry-based cytotoxicity assays).

TCR Sequencing, Transfer into Lentiviral Vectors, and Transduction of T Cells

Spliceosome neoantigen-specific TCR beta and alpha chains were sequenced by next-generation sequencing (Adaptive Biotechnologies, Takara Bio). TCRs were constructed by pairing the sequences encoding the dominant TCR beta and alpha chains in each spliceosome neoantigen-specific T cell clone and including cysteine modifications and codon optimization. In the case of the SF3B1^{K700E} specific TCR, murine rather than human constant regions were utilized to enhance TCR expression. When two dominant TCR alpha chains were identified from a single clone, two TCRs were assembled (e.g., B1A1 and B1A2) and tested. TCR constructs were synthesized by GeneArt (Life Technologies) and cloned into the pRRLSIN.cPPT.MSCV.WPRE lentiviral vector (LV) that included the RQR8 selection marker by restriction digestion and ligation.

CD8+ T cells immunomagnetically purified from normal donor PBMC were activated with Dynabeads Human T-Activator CD3/CD28 (ThermoFisher Scientific) in 50 IU/mL IL-2 for 24 hours, and in some experiments, the endogenous TCR alpha and beta chains were knocked out using CRISPR/Cas9 technology. Three days after CRISPR, T cells were enriched for CD3-TCR knockout cells, then transduced with LV supernatant. Four days after transduction, T cells were stained with spliceosome neoantigen pHLA tetramer and anti-CD8 monoclonal antibody (mAb). Spliceosome neoantigen tetramer+CD8+ T cells were sorted to >95% purity, expanded, and evaluated by flow cytometry and functional assays.

### Cell Lines

Epstein-Barr virus (EBV) transformed LCL were prepared and maintained in RPMI 1640, 10% fetal calf serum and 1% penicillin/streptomycin (LCL medium). Lenti-X 293T cells (Clontech) used in LV production were maintained in DMEM (Invitrogen) supplemented d with 10% FCS, 25 mM HEPES, 2 mM L-glutamine, 1% penicillin/streptomycin, and detached for passage using 0.05% trypsin-EDTA (Invitrogen). T cells were maintained in RPMI 1640, 10% human serum, 1% penicillin/streptomycin, 3 mM L-glutamine, and 50 μM β-mercaptoethanol (CTL medium).

### ⁵¹Chromium-Release Cytotoxicity Assays (CRA)

Cytotoxicity was measured in short-term (4-hour) assays using 51Cr-labeled target cells. Briefly, target cells were labeled with ⁵¹Cr overnight (cell lines) or for 6 hours (primary leukemia) at 37° C. and 5% CO₂. Effector cells were added to labeled target cells and incubated for 4 hours. After co-incubation, supernatant was harvested for γ-counting. Specific lysis was calculated using a standard formula. Targets used included autologous LCL with and without peptides in varying concentrations. All targets were washed to remove excess 51Cr and residual interferon-γ or peptide before co-culture initiation.

### Flow Cytometry-Based Degranulation Assay

In cytotoxic degranulation (CD107a) assays, effector T cells and stimulator cells (autologous LCL with and without peptide; primary malignant myeloid cells or iPSC-derived hematopoietic cell lines that were genotypically positive for SF3B1^{K700E} and HLA-B*40:01, or negative for either the mutation or HLA) were washed and plated in a 1:2 E:T ratio in LCL medium with GolgiStop transport inhibitor (BD Biosciences) and PE-conjugated anti-CD107a monoclonal antibody (mAb). For primary patient stimulator cells, cells were thawed, washed, suspended in LCL medium supplemented with 500 U/mL interferon-γ, and incubated for at least 24 hours at 37° C. prior to setting up co-culture with effector cells. Effectors and targets were co-incubated for 5 hours at 37° C. Cells were then washed and stained with mAb against CD8, CD19 or CD33, and DAPI.

### Next-generation Sequencing of TCR Genes

Survey-level sequencing of the variable V-J or V-D-J regions of the TRA and TRB genes (Adaptive Biotechnologies, Seattle, WA) was performed on genomic DNA extracted from ˜6×10⁴ T cells from SF3B1^{K700E} specific T cell clones. These regions encode CDR3 of the TCR a and B chains (i.e., the hypervariable amino acid sequences responsible for contact with the cognate peptide). The coverage per sample was >10×. The CDR1 and CDR2 sequences were determined using IMGT/V-QUEST software (http://www.imgt.org/IMGT_vquest/input).

### Flow Cytometry Monoclonal Antibodies and Instruments

Flow cytometry was performed on a 5-laser (355 nm, 405 nm, 488 nm, 552 or 532 nm and 628 or 640 nm) Fortessa X50 or Symphony instrument (BD). All data was analyzed with FlowJo software (Tree Star). All monoclonal antibodies (mAbs) used for flow cytometry were mouse anti-human.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

TABLE 8
In silico HLA binding and CTL epitope predictions for candidate spliceosome neoantigens and HLA binding predictions
for equivalent wild-type (wt) peptides.

| | | | | | | | % | Total | | | |
|---|---|---|---|---|---|---|---|---|---|---|---|
| | | | | ann_ | smm_ | | Rank_ | cri- | | | |
| MHC | Peptide | Identity | length | ic50 | ic50 | Aff(nM) | EL | teria | MHC | TAP | Cle |
| HLA- | QEVRTISAL | sf3b1_k700e | 9 | 14.08 | 54.8150734 | 14.14 | 0.034 | 4 | 0.686 | 0.861 | 0.95859 |
| B*40:01 | SEQ ID NO: 1 | | | | | | | | | | |
| HLA- | QEVRTISAL | sf3b1_k700e | 9 | 576.54 | 2106.68693 | 278.22 | 0.211 | 2 | 0.314 | 0.861 | 0.95859 |
| B*44:02 | SEQ ID NO: 1 | | | | | | | | | | |
| HLA- | QEVRTISAL | sf3b1_k700e | 9 | 187.17 | 524.191531 | 250.22 | 0.174 | 3 | 0.358 | 0.861 | 0.95859 |
| B*44:03 | SEQ ID NO: 1 | | | | | | | | | | |
| HLA- | RTPDSHHSR | srsf2_p95t | 9 | NA | NA | 348.32 | 0.235 | 2 | 0.536 | 1.662 | 0.97221 |
| A*33:03 | SEQ ID NO: 33 | | | | | | | | | | |
| HLA- | RLPDSHHSR | srsf2_p951 | 9 | NA | NA | 423.56 | 0.371 | 2 | 0.453 | 1.703 | 0.97623 |
| A*33:03 | SEQ ID NO: 35 | | | | | | | | | | |
| HLA- | GLVDEQQEV | sf3b1_k700e | 9 | 78.76 | 91.0143769 | 34.94 | 0.026 | 4 | 0.676 | 0.21 | 0.88853 |
| A*02:01 | SEQ ID NO: 36 | | | | | | | | | | |
| HLA- | FRLHNKPTF | u2af1_s34f | 9 | 40.59 | 29.3879979 | 163.59 | 0.048 | 4 | 0.322 | 2.657 | 0.8579 |
| C*07:02 | SEQ ID NO: 38 | | | | | | | | | | |
| HLA- | RLPDSHHSRR | srsf2_p951 | 10 | 452.04 | 594.565903 | 453.71 | 0.822 | 3 | 0.365 | 1.676 | 0.93525 |
| A*03:01 | SEQ ID NO: 40 | | | | | | | | | | |
| HLA- | FRLHNKPTF | u2af1_s34f | 9 | 321.09 | 88.7503348 | 348.56 | 0.059 | 4 | 0.256 | 2.657 | 0.8579 |
| C*07:01 | SEQ ID NO: 38 | | | | | | | | | | |
| HLA- | FRLHNKPTF | u2af1_s34f | 9 | 126.57 | 366.277297 | 546.44 | 0.102 | 3 | 0.087 | 2.657 | 0.8579 |
| C*06:02 | SEQ ID NO: 38 | | | | | | | | | | |
| HLA- | RTPDSHHSR | srsf2_p95t | 9 | 398.82 | 394.729878 | 660.89 | 0.324 | 3 | 0.378 | 1.662 | 0.97221 |
| A*11:01 | SEQ ID NO: 33 | | | | | | | | | | |
| HLA- | VQMARYGRL | srsf2_p951 | 9 | 544.71 | 954.00357 | 251.91 | 0.973 | 2 | 0.345 | 1.289 | 0.46104 |
| B*08:01 | SEQ ID NO: 42 | | | | | | | | | | |
| HLA- | QQEVRTISAL | sf3b1_k700e | 10 | 209.27 | 2377.00448 | 98.86 | 0.434 | 3 | 0.178 | 0.738 | 0.95799 |
| B*40:01 | SEQ ID NO: 44 | | | | | | | | | | |
| HLA- | QQEVRTISAL | sf3b1_k700e | 10 | 122.02 | 182.915286 | 143.17 | 2.592 | 3 | 0.253 | 0.738 | 0.95799 |
| B*15:01 | SEQ ID NO: 44 | | | | | | | | | | |
| HLA- | VQMARYGRL | srsf2_p951 | 9 | 350.92 | 553.350109 | 487.17 | 2.629 | 2 | 0.324 | 1.289 | 0.46104 |
| B*15:01 | SEQ ID NO: 42 | | | | | | | | | | |
| HLA- | DEQQEVRTI | sf3b1_k700e | 9 | 1183.01 | 186.037288 | 1861.38 | 0.233 | 2 | 0.213 | 0.098 | 10.33382 |
| B*44:02 | SEQ ID NO: 46 | | | | | | | | | | |

| | | | | | | | wt | wt | wt | |
|---|---|---|---|---|---|---|---|---|---|---|
| | | % | CTL | | %Rank_ | | binds | binds | binds | Meets |
| MHC | Comb | Rank | epitope? | wt pept | EL | Aff(nM) | (BA)? | (EL)? | (either)? | criteria? |
| HLA- | 0.92321 | 0.1 | YES | QKVRTISAL | 2.676 | 10334.24 | no | no | no | YES |
| B*40:01 | | | | SEQ ID NO: 2 | | | | | | |
| HLA- | 0.55121 | 0.8 | YES | QKVRTISAL | 8.304 | 24565.61 | no | no | no | YES |
| B*44:02 | | | | SEQ ID NO: 2 | | | | | | |
| HLA- | 0.59521 | 0.8 | YES | QKVRTISAL | 7.222 | 25391.17 | no | no | no | YES |
| B*44:03 | | | | SEQ ID NO: 2 | | | | | | |
| HLA- | 0.7963 | 0.3 | YES | RPPDSHHSR | 0.921 | 2236.08 | no | YES | YES | no |
| A*33:03 | | | | SEQ ID NO: 34 | | | | | | |
| HLA- | 0.71523 | 0.8 | YES | RPPDSHHSR | 0.921 | 2236.08 | nc | YES | YES | no |
| A*33:03 | | | | SEQ ID NO: 34 | | | | | | |
| HLA- | 0.88117 | 0.8 | YES | GLVDEQQKV | 0.044 | 110.58 | YES | YES | YES | no |
| A*02:01 | | | | SEQ ID NO: 37 | | | | | | |
| HLA- | 0.58145 | 0.8 | YES | SRLHNKPTF | 0.122 | 1050 | no | YES | YES | no |
| C*07:02 | | | | SEQ ID NO: 39 | | | | | | |
| HLA- | 0.61733 | 1.5 | no | RPPDSHHSRR | 6.804 | 20911.41 | no | no | no | no |
| A*03:01 | | | | SEQ ID NO: 41 | | | | | | |
| HLA- | 0.51545 | 1.5 | no | SRLHNKPTF | 0.079 | 1258.24 | no | YES | YES | no |
| C*07:01 | | | | SEQ ID NO: 39 | | | | | | |
| HLA- | 0.34645 | 2 | no | SRLHNKPTF | 0.202 | 2543.01 | no | YES | YES | no |
| C*06:02 | | | | SEQ ID NO: 39 | | | | | | |
| HLA- | 0.6383 | 3 | no | RPPDSHHSR | 4.539 | 19697.55 | no | no | no | no |
| A*11:01 | | | | SEQ ID NO: 34 | | | | | | |
| HLA- | 0.48096 | 3 | no | VQMARYGRP | 31.279 | 14249.52 | no | no | no | no |
| B*08:01 | | | | SEQ ID NO: 43 | | | | | | |
| HLA- | 0.412 | 3 | no | QQKVRTISAL | 3.974 | 5776.33 | no | no | no | no |
| B*40:01 | | | | SEQ ID NO: 45 | | | | | | |
| HLA- | 0.487 | 6 | no | QQKVRTISAL | 1.294 | 106.13 | YES | no | YES | no |
| B*15:01 | | | | SEQ ID NO: 45 | | | | | | |
| HLA- | 0.45996 | 7 | no | VQMARYGRP | 18.957 | 9556.94 | nc | no | no | no |
| B*15:01 | | | | SEQ ID NO: 43 | | | | | | |
| HLA- | 0.29056 | 9 | no | DEQQKVRTI | 0.198 | 2070.26 | no | YES | YES | no |
| B*44:02 | | | | SEQ ID NO: 47 | | | | | | |

TABLE 9
Analysis of set of known immunogenic and naturally processed and presented epitopes using netCTLpan.

| | | | | | netCTLpan Results | | | | | |
|---|---|---|---|---|---|---|---|---|---|---|
| Parent | | | | Known | | | | | Combined | % |
| gene | Peptide | mer | Input amino acid sequence | HLA | Alelle | MHC | TAP | Cle | Score | Rank |
| SMCY | FIDSYICQV | 9 | LRKNHSSAQFIDSYICQVCSRGDEDNK | HLA- | HLA- | 0.895 | 0.123 | 0.975949 | 1.117664 | 0.05 |
| | SEQ ID NO: | | SEQ ID NO: 49 | A*02:01 | A*02:01 | | | | | |
| | 48 | | | | | | | | | |
| CBFB- | REEMEVHEL | 9 | EDRDRSHREEMEVHELEKSKRALET | HLA- | HLA- | 0.688 | 0.896 | 0.961935 | 0.926835 | 0.1 |
| MYH11 | SEQ ID NO: | | SEQ ID NO: 51 | B*40:01 | B*40:01 | | | | | |
| type A | 50 | | | | | | | | | |
| WT1 | RMFPNAPYL | 9 | PSQASSGQARMFPNAPYLPSCLESQPAI | HLA- | HLA- | 0.805 | 1.586 | 0.977494 | 1.064586 | 0.1 |
| | SEQ ID NO: | | SEQ ID NO: 53 | A*02:01 | A*02:01 | | | | | |
| | 52 | | | | | | | | | |
| BCL2A1 | KEFEDDIINW | 10 | TARTLFNQVMEKEFEDDIINWGRIVTIF | HLA- | HLA- | 0.651 | 0.801 | 0.40699 | 0.762598 | 0.15 |
| | SEQ ID NO: | | SEQ ID NO: 55 | B*44:03 | B*44:03 | | | | | |
| | 54 | | | | | | | | | |
| BCL2A1 | DYLQYVLQI | 9 | MTDCEFGYIYRLAQDYLQYVLQIPQPGSG | HLA- | HLA- | 0.619 | 0.452 | 0.906645 | 0.834295 | 0.2 |
| | SEQ ID NO: | | PS | A*24:02 | A*24:02 | | | | | |
| | 56 | | SEQ ID NO: 57 | | | | | | | |
| HMHA1 | VLHDDLLEA | 9 | AEGLEKLKECVLHDDLLEARRPRAHECLG | HLA- | HLA- | 0.696 | −0.431 | 0.95649 | 0.900435 | 0.8 |
| (HA-1) | SEQ ID NO: | | SEQ ID NO: 59 | A*02:01 | A*02:01 | | | | | |
| | 58 | | | | | | | | | |
| HEATR1 | ISKERAEAL | 9 | FLFDTQHFISKERAEALMMPLVDQLE | HLA- | HLA- | 0.424 | 0.911 | 0.697728 | 0.603764 | 1.5 |
| | SEQ ID NO: | | SEQ ID NO: 61 | B*08:01 | B*08:01 | | | | | |
| | 60 | | | | | | | | | |
| NPM1 | CLAVEEVSL | 9 | NCFRMTDQEAIQDLCLAVEEVSLRK | HLA- | HLA- | 0.351 | 0.998 | 0.93904 | 0.587234 | 4 |
| type A | SEQ ID NO: | | SEQ ID NO: 63 | A*02:01 | A*02:01 | | | | | |
| | 62 | | | | | | | | | |

1. An engineered T cell receptor (TCR) or a fragment thereof that specifically binds SF3B1^{K700E}, an epitope of SF3B1^{K700E}, or an SF3B1^{K700E} epitope/human leukocyte antigen (HLA) complex, comprising:
(a) an alpha chain variable region comprising one, two, or three complementarity determining regions (CDRs) having amino acid sequences set forth in SEQ ID NOs: 12-14; and/or
(b) a beta chain variable region comprising one, two, or three CDRs having amino acid sequences set forth in SEQ ID NOs: 6-8.

2. The TCR or fragment thereof of claim 1, wherein the epitope of SF3B1^{K700E} has an amino acid sequence set forth in SEQ ID NO: 1.

3. The TCR or fragment thereof of claim 1 or claim 2, wherein the HLA is HLA-B*40:01.

4. The TCR or fragment thereof of any one of claims 1-3, comprising an alpha chain variable region comprising an amino acid sequence that is at least about 80% identical to SEQ ID NO: 32; and/or a beta chain variable region comprising an amino acid sequence that is at least about 80% identical to SEQ ID NO: 31.

5. The TCR or fragment thereof of any one of claims 1-4, comprising an alpha chain constant region comprising an amino acid sequence that is at least about 80% identical to SEQ ID NO: 20 or SEQ ID NO: 22; and/or a beta chain constant region comprising an amino acid sequence that is at least about 80% identical to SEQ ID NO: 16 or SEQ ID NO: 18.

6. The TCR or fragment thereof of any one of claims 1-5, comprising an amino acid sequence that is at least about 80% identical to SEQ ID NO: 25.

7. A nucleic acid comprising a nucleotide sequence encoding the TCR or fragment thereof of any one of claims 1-6.

8. The nucleic acid of claim 7, wherein the nucleotide sequence is codon-optimized.

9. A nucleic acid comprising a nucleotide sequence that is at least 80% identical to a nucleotide sequence selected from the group consisting of SEQ ID NOs: 3, 4, 9, 10, 15, 17, 19, 21, 23, and 24.

10. A vector comprising the nucleic acid of any one of claims 7-9.

11. The vector of claim 10, wherein the vector is a plasmid, an adenoviral vector, an adeno-associated viral vector, a retroviral vector, or a lentiviral vector.

12. A virus comprising the nucleic acid of any one of claims 7-9.

13. The virus of claim 12, wherein the virus is an adenovirus, an adeno-associated virus, a retrovirus, a lentivirus, or a phage.

14. A composition comprising the vector of claim 10 or claim 11, or the virus of claim 12 or claim 13.

15. The composition of claim 14, further comprising a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

16. A host cell expressing the TCR or fragment thereof of any one of claims 1-6, comprising the nucleic acid of any one of claims 7-9, and/or comprising the vector of claim 10 or claim 11.

17. The host cell of claim 16, wherein the host cell is a T cell.

18. The host cell of claim 17, wherein the T cell is a CD8+ T cell.

19. The host cell of any one of claims 16-18, wherein the host cell is an autologous cell.

20. The host cell of any one of claims 16-18, wherein the host cell is an allogeneic cell.

21. The host cell of any one of claims 16-20, wherein the host cell is differentiated from an embryonic stem cell (ESC) or an induced pluripotent stem cell (iPSC).

22. The host cell of any one of claims 16-20, wherein the host cell is a primary cell.

23. The host cell of any one of claims 16-22, wherein the host cell is modified to have reduced or eliminated expression of an endogenous TCR.

24. The host cell of any one of claims 16-23, wherein the host cell has knockout of one or more endogenous TCR genes.

25. The host cell of claim 24, wherein the one or more endogenous TCR genes are selected from the group consisting of TRAC, TRBC1, and TRBC2.

26. The host cell of any one of claims 16-25, wherein the host cell is modified to have reduced or eliminated expression of one or more major histocompatibility complex (MHC) class I molecules selected from the group consisting of HLA-A, HLA-B, and HLA-C.

27. The host cell of any one of claims 16-26, wherein the host cell is modified to express HLA-E and/or HLA-G.

28. A pharmaceutical composition, comprising the host cell of any one of claims 16-27.

29. A method of treating an SF3B1^{K700E}-positive cancer in a subject in need thereof, comprising administering to the subject the host cell of any of claims 16-27, or the pharmaceutical composition of claim 28.

30. The method of claim 29, wherein treating an SF3B1^{K700E}-positive cancer comprises inhibiting cancer cell growth, reducing the number of cancer cells, slowing the progression of cancer, decreasing the likelihood of recurrence, or reducing one or more symptoms associated with the cancer.

31. The method of claim 29 or claim 30, wherein the cancer is a hematologic malignancy.

32. The method of claim 31, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

33. A pharmaceutical composition comprising the host cell of any one of claims 16-27 for use in a method of treating an SF3B1^{K700E}-positive cancer in a subject in need thereof.

34. The pharmaceutical composition of claim 33, wherein treating an SF3B1^{K700E}-positive cancer comprises inhibiting cancer cell growth, reducing the number of cancer cells, slowing the progression of cancer, decreasing the likelihood of recurrence, or reducing one or more symptoms associated with the cancer.

35. The pharmaceutical composition of claim 33 or claim 34, wherein the cancer is a hematologic malignancy.

36. The pharmaceutical composition of claim 35, wherein the hematologic malignancy is selected from the group consisting of myeloid neoplasm, MDS, myeloproliferative/myelodysplastic syndromes, ALL, CLL, AML, CML, bcCML, B-ALL, T-ALL, T-cell lymphoma, and B-cell lymphoma.

37. Use of the host cell of any of claims 16-27 in the manufacture of a medicament for treating an SF3B1^{K700E}-positive cancer in a subject in need thereof.

38. The use of claim 37, wherein treating an SF3B1^{K700E}-positive cancer comprises inhibiting cancer cell growth, reducing the number of cancer cells, slowing the progression of cancer, decreasing the likelihood of recurrence, or reducing one or more symptoms associated with the cancer.

39. The use of claim 37 or claim 38, wherein the cancer is a hematologic malignancy.

40. The use of claim 39, wherein the hematologic malignancy is selected from the group consisting of myeloid neoplasm, MDS, myeloproliferative/myelodysplastic syndromes, ALL, CLL, AML, CML, bcCML, B-ALL, T-ALL, T-cell lymphoma, and B-cell lymphoma.
